(19) United States
(12) Patent Application Publication
(10) Pub. No.: US 2023/0277305 A1
(43) Pub. Date: Sep. 7, 2023

(54) DOCKING STATION FOR A TRANSCATHETER HEART VALVE

(71) Applicant: EDWARDS LIFESCIENCES CORPORATION, Irvine, CA (US)

(72) Inventors: Shahram Zamani, Newport Coast, CA (US); Alison Louise Rodriguez, Irvine, CA (US); Anthony Michael Romero, Corona, CA (US)

(21) Appl. No.: 18/314,752

(22) Filed: May 9, 2023

Related U.S. Application Data
(63) Continuation of application No. PCT/US2021/058588, filed on Nov. 9, 2021.
(60) Provisional application No. 63/111,879, filed on Nov. 10, 2020.

(51) Int. Cl. A61F 2/24

(52) U.S. Cl. CPC A61F 2/2409; A61F 2230/0006; A61F 2230/0008; A61F 2230/0034; A61F 2250/0098

(57) ABSTRACT

Docking stations are configured to retain and position a transcatheter heart valve in a circulatory system. The docking stations can comprise an expandable frame. The docking stations can include an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion. The docking station can be configured to adapt a native tricuspid valve to accept a smaller transcatheter heart valve.

### CROSS-REFERENCE TO RELATED APPLICATION

This application is a continuation of PCT Patent Application No. PCT/US2021/058588, filed on Nov. 9, 2021, which application claims the benefit of U.S. Provisional Application No. 63/111,879 filed on Nov. 10, 2020, each of these applications being incorporated by this specific reference in its entirety.

### FIELD OF THE INVENTION

The present invention relates to heart valves and, in particular, a docking station or docking stent including a transcatheter heart valve (THV) or for use in implanting a transcatheter heart valve.

### BACKGROUND OF THE INVENTION

Prosthetic heart valves can be used to treat cardiac valvular disorders. The native heart valves (the aortic, pulmonary, tricuspid and mitral valves) function to prevent backward flow or regurgitation, without preventing forward flow. These heart valves can be rendered less effective by congenital, inflammatory, or infectious conditions. Such conditions can eventually lead to serious cardiovascular compromise or death. For many years the definitive treatment for such disorders was the surgical repair or replacement of the valve during open heart surgery.

A transcatheter technique can also be used for introducing and implanting a prosthetic heart valve using a catheter in a manner that is less invasive than open heart surgery. In this technique, a prosthetic valve can be mounted in a crimped state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted. Alternatively, the valve can have a resilient, self-expanding stent or frame that expands the valve to its functional size when it is advanced from a delivery sheath at the distal end of the catheter.

Transcatheter heart valves (THVs) may be appropriately sized to be placed inside most native aortic valves. However, with larger native valves, blood vessels, and grafts, aortic transcatheter valves might be too small to secure into the larger implantation or deployment site. In this case, the transcatheter valve may not be large enough to sufficiently expand inside the native valve or other implantation or deployment site to be secured in place.

### SUMMARY

According to an exemplary embodiment of the present disclosure, an expandable frame for a docking station configured to retain and position a transcatheter heart valve in a circulatory system includes an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion. The expandable frame includes a plurality of struts extending between first apices at the first end portion to second apices at the second end portion, wherein one of the first apices and the second apices are contoured radially inward.

According to another exemplary embodiment of the present disclosure, an expandable frame for a docking station configured to retain and position a transcatheter heart valve in a circulatory system includes an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion. The expandable frame includes a plurality of struts extending between first apices at the first end portion to second apices at the second end portion, wherein the plurality of struts include first end strut portions defining the first end portion of the frame, second end strut portions defining the second end portion of the frame, and central strut portions defining the waist portion of the frame. The central strut portions have a cross-sectional area greater than a cross-sectional area of the first and second end strut portions.

According to another exemplary embodiment of the present disclosure, an expandable frame for a docking station configured to retain and position a transcatheter heart valve in a circulatory system includes an enlarged first end portion having an elliptical first outer radial portion with a first major lateral dimension, an enlarged second end portion having an elliptical second outer radial portion with a second major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion.

According to another exemplary embodiment of the present disclosure, an expandable frame for a docking station configured to retain and position a transcatheter heart valve in a circulatory system includes an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion. A first axial length from an axial midpoint of the waist portion to an edge of the first end portion is greater than a second axial length from the axial midpoint of the waist portion to an edge of the second end portion.

According to another exemplary embodiment of the present disclosure, an expandable frame for a docking station configured to retain and position a transcatheter heart valve in a circulatory system includes an enlarged first end portion having a first outer radial portion with a first major lateral dimension, an enlarged second end portion having a second outer radial portion with a second major lateral dimension greater than the first major lateral dimension, and a narrowed central waist portion having an inner radial portion with a third major lateral dimension smaller than the first and second major lateral dimensions. A retaining portion is at least partially defined by at least one of the first and second end portions, and a valve seat is at least partially defined by the waist portion.

According to another exemplary embodiment of the present disclosure, an expandable frame for a docking station configured to retain and position a transcatheter heart valve in a circulatory system includes an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions, wherein the first outer radial portion has a cross-sectional shape different than a cross-sectional shape of at least one of the second outer radial portion and the inner radial portion. A retaining portion is at least partially defined by at least one of the first and second end portions, and a valve seat is at least partially defined by the waist portion.

According to another exemplary embodiment of the present disclosure, an expandable frame for a docking station configured to retain and position a transcatheter heart valve in a circulatory system includes a first end flange portion extending radially outward to a first outer radial portion with a first major lateral dimension, an enlarged second end portion extending radially outward to a second outer radial portion with a second major lateral dimension, and a narrowed axially extending central waist portion having a third major lateral dimension smaller than the first and second major lateral dimensions, with the first and second end flange portions extending substantially perpendicularly to a central axis of the frame when the frame is in an unconstrained condition. A retaining portion is at least partially defined by at least one of the first and second end flange portions, and a valve seat is at least partially defined by the waist portion.

According to another exemplary embodiment of the present disclosure, a method of deploying a docking station to a tricuspid valve of a human heart is contemplated. In the exemplary method, an outer catheter is guided through a right atrium and tricuspid valve, and into a right ventricle. An inner catheter is guided within the outer catheter to extend an open end of the inner catheter to or beyond an open end of the outer catheter. The outer and inner catheters are adjusted to align the open end of the inner catheter with an intended deployment site for a docking station. A compressed docking station is guided through and out of the inner catheter, with the docking station expanding into retaining and sealing engagement with the deployment site.

### BRIEF DESCRIPTION OF THE DRAWINGS

Further understanding of the nature and advantages of the disclosed inventions can be obtained from the following description and claims, particularly when considered in conjunction with the accompanying drawings in which like parts bear like reference numerals.

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures may be drawn to scale for some embodiments, the figures are not necessarily drawn to scale for all embodiments. Embodiments of the present disclosure will be described and explained with additional specificity and detail through the use of the accompanying drawings.

FIG. 1 is a cutaway view of the human heart in a diastolic phase;

FIG. 2 is a cutaway view of the human heart in a systolic phase;

FIG. 3 is a cutaway view of the human heart with an exemplary embodiment of a docking station and transcatheter heart valve (THV) positioned in the tricuspid valve annulus;

FIG. 4A is a schematic illustration of a compressed docking station being positioned at a native annulus of a circulatory system;

FIG. 4B is a schematic illustration of the docking station of FIG. 4A expanded to set the position of the docking station in the circulatory system;

FIG. 4C is a schematic illustration of an expandable transcatheter heart valve being positioned in the docking station illustrated by FIG. 4B;

FIG. 4D is a schematic illustration of the transcatheter heart valve of FIG. 4C expanded to set the position of the heart valve in the docking station;

FIG. 5A is a perspective view of an exemplary embodiment of an expandable frame for a docking station;

FIG. 5B is a side elevational view of the expandable frame of FIG. 5A;

FIG. 5C is a front elevational view of the expandable frame of FIG. 5A;

FIG. 5D is a top plan view of the expandable frame of FIG. 5A;

FIG. 5E is a front view of an exemplary lattice sheet for an expandable frame;

FIG. 5F is a side view of the lattice sheet of FIG. 5E;

FIG. 6A is a side view of another exemplary expandable frame for a docking station;

FIG. 6B is a top view of the expandable frame of FIG. 6A;

FIG. 6C is a schematic illustration of the docking station of FIG. 6A, shown being positioned at a native annulus of a circulatory system;

FIG. 6D is a schematic illustration of the docking station of FIG. 6A expanded to set the position of the docking station in the circulatory system;

FIG. 6E is a schematic illustration of an expandable transcatheter heart valve being positioned in the docking station illustrated by FIG. 6D;

FIG. 6F is a schematic illustration of the transcatheter heart valve of FIG. 6E expanded to set the position of the heart valve in the docking station;

FIG. 7 is a side view of another exemplary expandable frame for a docking station;

FIG. 8 is a partial view of an exemplary expandable frame, showing exemplary first end, second end, and central cells of the expandable frame;

FIG. 8A is a cross-sectional view of a first end portion of a strut of the expandable frame of FIG. 8, taken along the plane indicated by lines 8A-8A of FIG. 8;

FIG. 8B is a cross-sectional view of a second end portion of a strut of the expandable frame of FIG. 8, taken along the plane indicated by lines 8B-8B of FIG. 8;

FIG. 8C is a cross-sectional view of a central portion of a strut of the expandable frame of FIG. 8, taken along the plane indicated by lines 8C-8C of FIG. 8;

FIG. 9A is a schematic view of a tricuspid valve region of the human heart;

FIG. 9B is a side view of an exemplary embodiment of an expandable frame for a docking station, shown implanted in a tricuspid valve region of the human heart;

FIG. 9C is a side view of an exemplary embodiment of an expandable frame for a docking station, shown implanted in a tricuspid valve region of the human heart;

FIG. 9D is a side view of an exemplary embodiment of an expandable frame for a docking station, shown implanted in a tricuspid valve region of the human heart;

FIG. 10A is a side elevational schematic view of an exemplary embodiment of an expandable frame for a docking station;

FIG. 10B is a front elevational schematic view of the expandable frame of FIG. 10A;

FIG. 10C is a top plan schematic view of the expandable frame of FIG. 10A;

FIG. 10D is an upper perspective schematic view of the expandable frame of FIG. 10A;

FIG. 10E is an upper perspective schematic view of another exemplary embodiment of an expandable frame for a docking station;

FIG. 10F is a side elevational schematic view of the expandable frame of FIG. 10E;

FIG. 10G is an upper perspective schematic view of another exemplary embodiment of an expandable frame for a docking station;

FIG. 10H is a top plan schematic view of the expandable frame of FIG. 10G;

FIG. 11 is a side view of an exemplary expandable frame for a docking station;

FIG. 12 is a side view of another exemplary expandable frame for a docking station;

FIGS. 13A - 13H are exemplary cross-sectional views of the first end portions, the second end portions and the waist portions of the expandable frames of FIGS. 11 and 12;

FIG. 14 is a side view of an exemplary expandable frame for a docking station;

FIG. 15 is a side view of another exemplary expandable frame for a docking station;

FIGS. 16A - 16H are exemplary cross-sectional views of the first end portions, the second end portions and the waist portions of the expandable frames of FIGS. 14 and 15;

FIG. 17A is a schematic illustration of an exemplary docking station having a sealing portion at a central waist portion of the docking station body;

FIG. 17B is a schematic illustration of an exemplary docking station having a sealing portion at central and second end portions of the docking station body;

FIG. 17C is a schematic illustration of an exemplary docking station having a sealing portion at central and first end portions of the docking station body;

FIG. 17D is a schematic illustration of an exemplary docking station having a sealing portion at central and first and second end portions of the docking station body;

FIG. 18A is a schematic illustration of an exemplary docking station having an exemplary sealing portion at a central waist portion of the docking station body;

FIG. 18B is a schematic illustration of an exemplary docking station having another exemplary sealing portion at a central waist portion of the docking station body;

FIG. 18C is a schematic illustration of an exemplary docking station having another exemplary sealing portion at a central waist portion of the docking station body;

FIG. 18D is a side view of an exemplary expandable frame for a docking station, including a sealing portion at a central waist portion of the frame;

FIG. 19A is a side view of an exemplary expandable frame for a docking station, including a sealing portion at a central waist portion of the frame;

FIG. 19B is a side view of an exemplary expandable frame for a docking station, including a sealing portion at central and second end portions of the frame;

FIG. 19C is a side view of an exemplary expandable frame for a docking station, including a sealing portion at central and first end portions of the frame;

FIG. 19D is a side view of an exemplary expandable frame for a docking station, including a sealing portion at central and first and second end portions of the frame;

FIG. 19E illustrates the expandable frame of FIG. 19A implanted in a circulatory system;

FIG. 19F illustrates the expandable frame of FIG. 19B implanted in a circulatory system;

FIG. 19G illustrates the expandable frame of FIG. 19C, implanted in a circulatory system;

FIG. 20A is a side view of an exemplary expandable frame for a docking station;

FIG. 20B is a top plan view of the expandable frame of FIG. 20A;

FIG. 20C is a side view of the expandable frame of FIG. 20A, including a sealing portion at a central waist portion of the frame;

FIG. 20D is a side view of an exemplary expandable frame for a docking station;

FIG. 20E is a side view of the expandable frame of FIG. 20A, including a sealing portion at a central waist portion of the frame;

FIGS. 21A - 21G illustrate an exemplary method for installing a THV through the superior vena cava for implantation at the tricuspid valve annulus; and

FIGS. 22A - 22E illustrate an exemplary method for installing a THV through the inferior vena cava for implantation at the tricuspid valve annulus.

### DETAILED DESCRIPTION

The following description refers to the accompanying drawings, which illustrate specific embodiments of the invention. Other embodiments having different structures and operation do not depart from the scope of the present invention. Exemplary embodiments of the present disclosure are directed to devices and methods for providing a docking station or landing zone for a transcatheter heart valve (“THV”). In some exemplary embodiments, docking stations for THVs are illustrated as being used within the right ventricle RV as a replacement tricuspid valve for a damaged or diseased native tricuspid valve TV. In other exemplary embodiments, docking stations may additionally or alternatively may be used in other areas of the anatomy, heart, or vasculature, such as the pulmonary valve, the aortic valve, and the mitral valve, or within the superior vena cava SVC and/or the inferior vena cava IVC. The docking stations described herein can be configured to compensate for the deployed THV being smaller and/or having a different geometrical shape than the space (e.g., anatomy/vasculature/etc.) in which the THV is to be placed.

It should be noted that various embodiments of docking stations and examples of THVs are disclosed herein, and any combination of these options may be made unless specifically excluded. For example, any of the docking stations devices disclosed, may be used with any type of valve, and/or any delivery system, even if a specific combination is not explicitly described. Likewise, the different constructions of docking stations and valves may be mixed and matched, such as by combining any docking station type/feature, valve type/feature, tissue cover, etc., even if not explicitly disclosed. In short, individual components of the disclosed systems may be combined unless mutually exclusive or otherwise physically impossible.

For the sake of uniformity, in these Figures and others in the application the docking stations are depicted such that the right atrium end is up, while the ventricular end or IVC end is down. These directions may also be referred to as “distal” as a synonym for up or the pulmonary bifurcation end, and “proximal” as a synonym for down or the ventricular end, which are terms relative to the physician’s perspective.

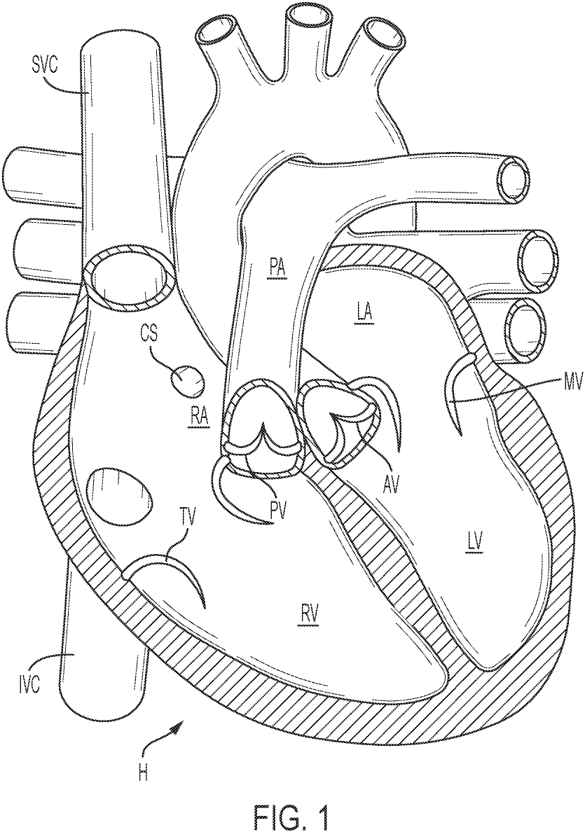

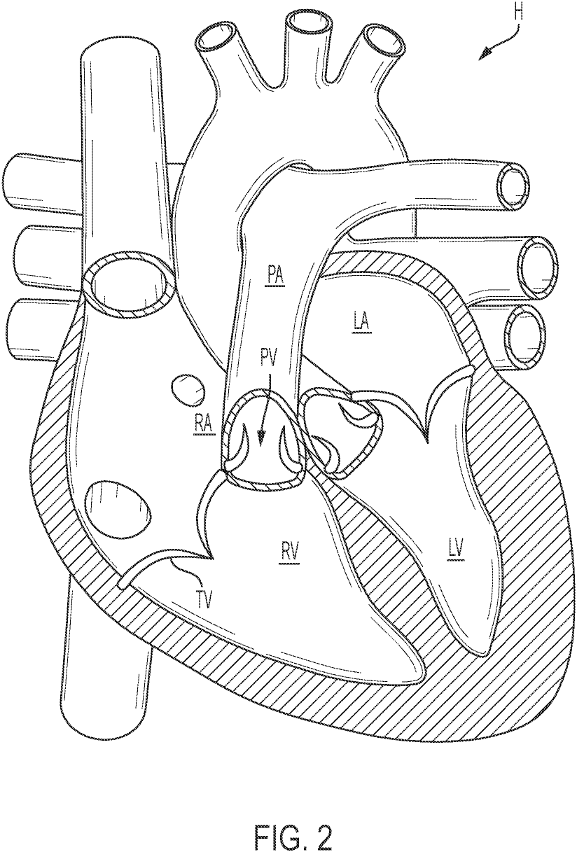

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta (not identified) and the pulmonary valve PV separates the right ventricle from the pulmonary artery PA. Each of these valves has flexible leaflets extending inward across the respective orifices that come together or “coapt” in the flowstream to form the one-way, fluid-occluding surfaces. The docking stations and valves of the present disclosure are described primarily with respect to the tricuspid valve. Therefore, anatomical structures of the right atrium RA and right ventricle RV will be explained in greater detail. It should be understood that the devices described herein may also be used in other areas, e.g., in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The coronary sinus CS is a collection of veins joined together to form a large vessel that collects deoxygenated blood from the heart muscle (myocardium), and delivers it to the right atrium RA. During the diastolic phase, or diastole, seen in FIG. 1, the venous blood that collects in the right atrium RA enters the right ventricle through the tricuspid valve TV by expansion of the right ventricle RV. In the systolic phase, or systole, seen in FIG. 2, the right ventricle RV contracts to force the venous blood through the pulmonary valve PV and pulmonary artery into the lungs, and the closed tricuspid valve prevents backflow of the blood into the right atrium RA.

Tricuspid valve diseases affecting the function of the tricuspid valve TV can be either functional or degenerative. In functional tricuspid regurgitation, there is high backflow or regurgitation of blood from the right ventricle RV through the tricuspid valve TV in the systolic phase, as the result of an enlarged right ventricle RV. This blood backflows or regurgitates into the right atrium RA, the inferior vena cava IVC, and the superior vena cava SVC. In tricuspid stenosis, which is typically a degenerative disease, there is decreased flow to the right ventricle as a result of a blockage or an enlarged right atrium RA. The traditional method of tricuspid valve replacement is performed through more invasive open heart surgery, due in part to the THV deployment challenges related to the anatomy of the tricuspid valve TV, including the soft, non-calcified state of the tricuspid valve annulus, the contours of the right atrium RA and right ventricle RV, and the presence of the chordae tendineae extending from the native tricuspid valve TV leaflets and anchored to the walls of the right ventricle RV.

In one exemplary embodiment, the devices described by the present disclosure are used to replace the function of a defective tricuspid valve. During systole, the leaflets of a normally functioning tricuspid valve TV close to prevent the venous blood from regurgitating back into the right atrium RA. According to an aspect of the present disclosure, a THV implanted at the native tricuspid valve annulus may prevent blood from backflowing from the right ventricle RV to the right atrium RA and into the inferior vena cave IVC and superior vena cava SVC during the systolic phase, and/or provide proper blood flow from the right atrium RA to the right ventricle RV in the diastolic phase.

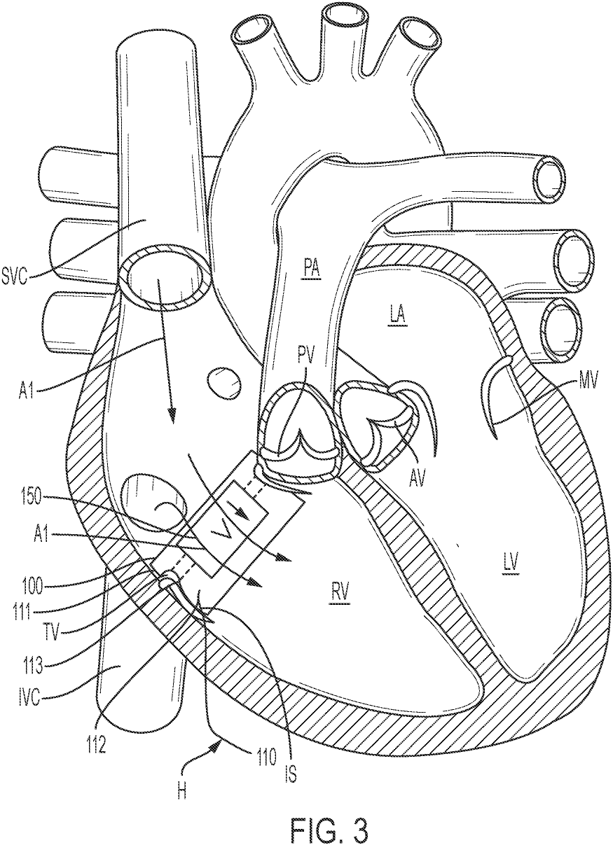

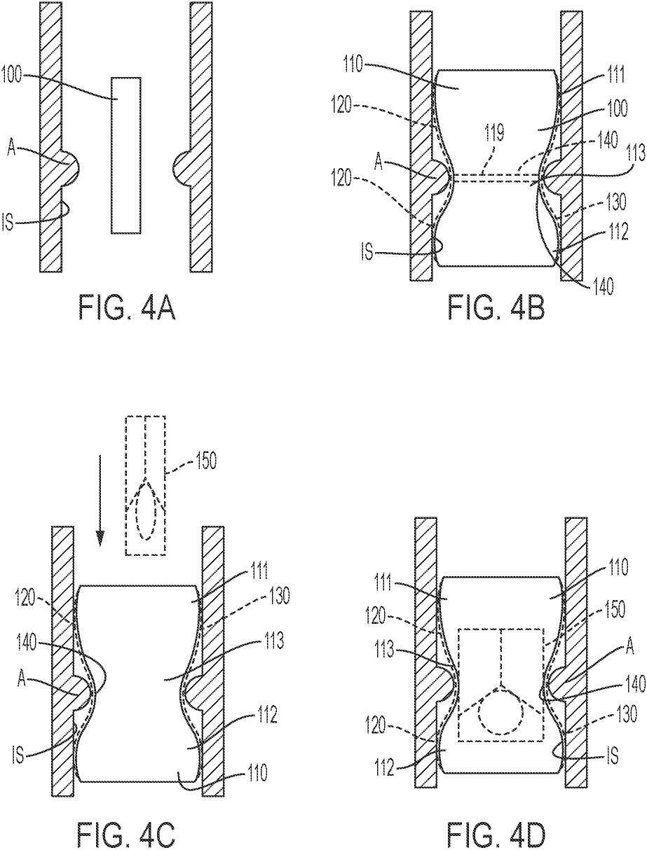

Referring to FIGS. 3 and 4A - 4D, in one exemplary embodiment an expandable docking station 100 configured to retain and position a transcatheter heart valve (THV) 150 at a native annulus in a circulatory system (e.g., at or near the native tricuspid valve TV annulus, as shown in FIG. 3). However, the expandable docking station 100 can be configured to retain and position a transcatheter heart valve (THV) 150 at any portion of a circulatory system, as is indicated by the generic representation of a portion of the vasculature illustrated by FIGS. 4A - 4D. In the examples of FIGS. 3 and 4A - 4D, the docking station includes an hourglass-shaped body 110 having an enlarged first or distal (e.g., inflow) end portion 111 sized and configured to be retained distal to the native annulus A (e.g., in the right atrium RA distal to the tricuspid valve TV annulus), an enlarged second or proximal (e.g., outflow) end portion 112 sized and configured to be retained proximal to the native annulus A (e.g., in the right ventricle RV proximal to the native tricuspid valve TV annulus), and a narrowed central portion or waist portion 113 sized and configured to align with and accommodate the native tricuspid valve TV.

In one exemplary embodiment, the proximal end of the end portion 111 and/or the distal end of the end portion 112 extends radially inward. This radial inward extension of the end portion 111 and/or end portion 112 can prevent the proximal end of the end portion 111 and/or the distal end of the end portion 112 from contacting the vasculature. The docking station body 110 may include a variety of suitable expandable structures. In an exemplary embodiment, the docking station body 110 includes an expandable lattice frame, as described in greater detail below.

The exemplary docking station 100 includes at least one retaining portion 120, disposed at one or both of the first and second end portions 111, 112 of the docking station body 110. The retaining portion 120 helps retain the docking station 100 and the valve 150 (described in greater detail below) at the implantation position or deployment site in the circulatory system. The retaining portion 120 can take a wide variety of different forms. As described herein, the retaining portion may include radially outward biased struts of a lattice frame docking station body. In some exemplary embodiments, the retaining portion 120 may additionally or alternatively include friction enhancing features that reduce or eliminate migration of the docking station 100. The friction enhancing features can take a wide variety of different forms. For example, the friction enhancing features may comprise barbs, spikes, and/or cloth with high friction properties on the retaining portions 120.

The exemplary docking station 100 further includes a valve seat 140 disposed on an inner diameter of the docking station body 110 to provide a supporting surface for implanting or deploying a valve 150 in the docking station after the docking station is implanted in the circulatory system. The valve seat 140 may be configured to position the valve 150 at a variety of locations along the docking station body 110, including, for example, aligned with and/or overlapping one or more of the first end portion 111, the second end portion 112, and the central waist portion 113. In an alternate embodiment, the docking station 100 and the valve 150 can be integrally formed, so that the valve seat 140 can be omitted. That is, the docking station 100 and the valve 150 can be deployed as a single device, rather than first deploying the docking station 100 and then deploying the valve 150 into the docking station. Any of the valve seats 140 described herein can be provided with an integrated valve 150.

The exemplary docking station 100 further includes at least one sealing portion 130, disposed at one or more of the first end portion 111, the second end portion 112, and the central waist portion 113 of the docking station body 110. The sealing portion(s) 130 provide a seal between the docking station 100 and an interior surface IS of the circulatory system, and between the valve 150 and the valve seat 140, for example, to minimize or prevent leakage around the closed valve 150 from the right ventricle RV to the right atrium RA in the systolic phase.

Expandable docking station 100 and valve 150 as described in the various embodiments herein are also representative of a variety of docking stations and/or valves that might be known or developed, e.g., a variety of different types of valves could be substituted for and/or used as valve 150 in the various docking stations.

FIGS. 3 and 4A - 4D illustrate operation of the docking stations 100 and valves 150 disclosed herein. In the illustrated example, the docking station 100 and valve 150 are deployed at the tricuspid valve TV, as shown in FIG. 3. However, in other arrangements, a docking station 100 and valve 150 including one or more of the features described herein may be deployed at any other suitable interior surface. For example, the docking station 100 and valve 150 may be deployed in the inferior vena cava IVC, the superior vena cava SVC, or at the pulmonary valve PV, the mitral valve MV, or the aortic valve AV.

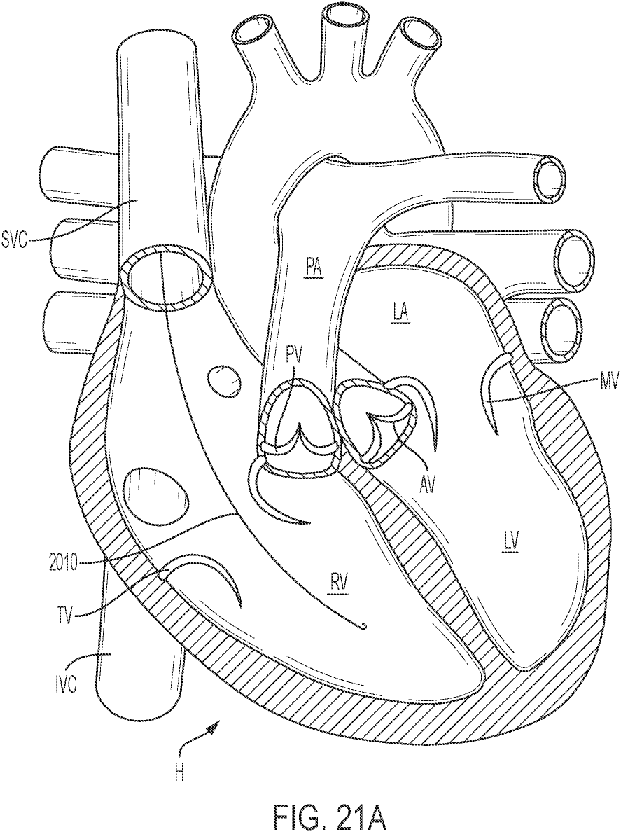

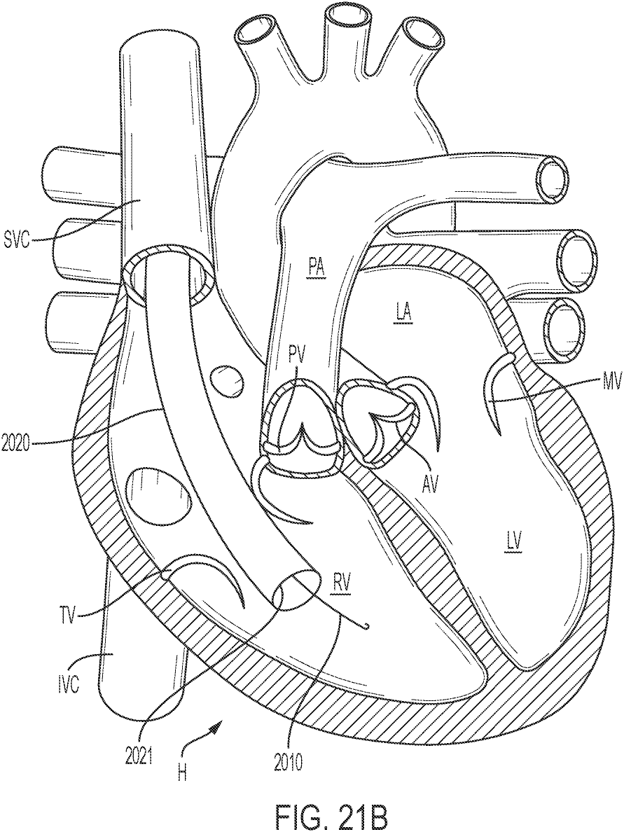

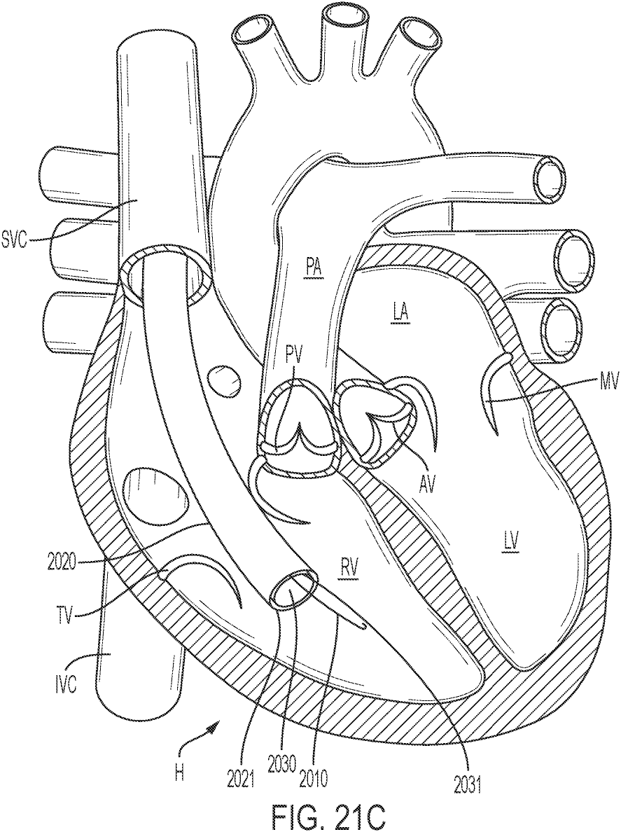

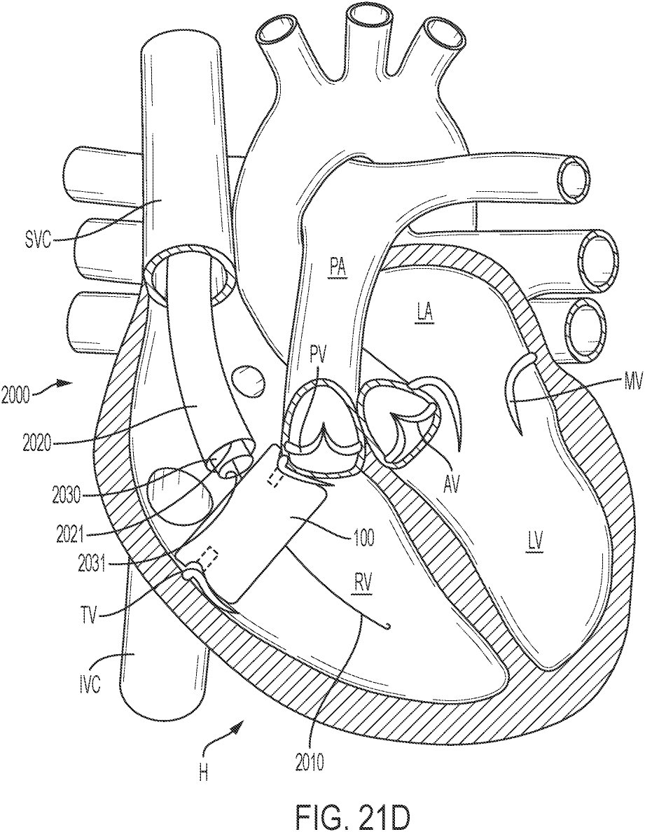

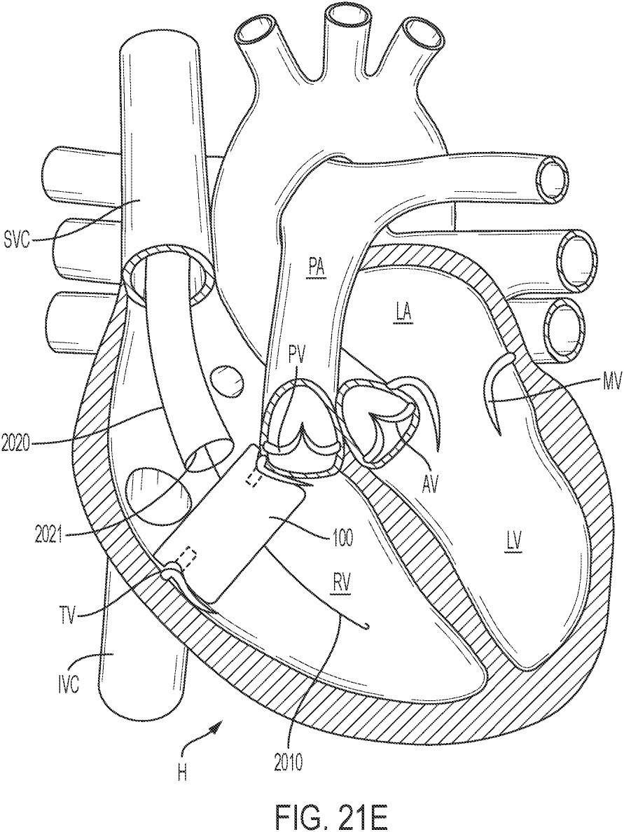

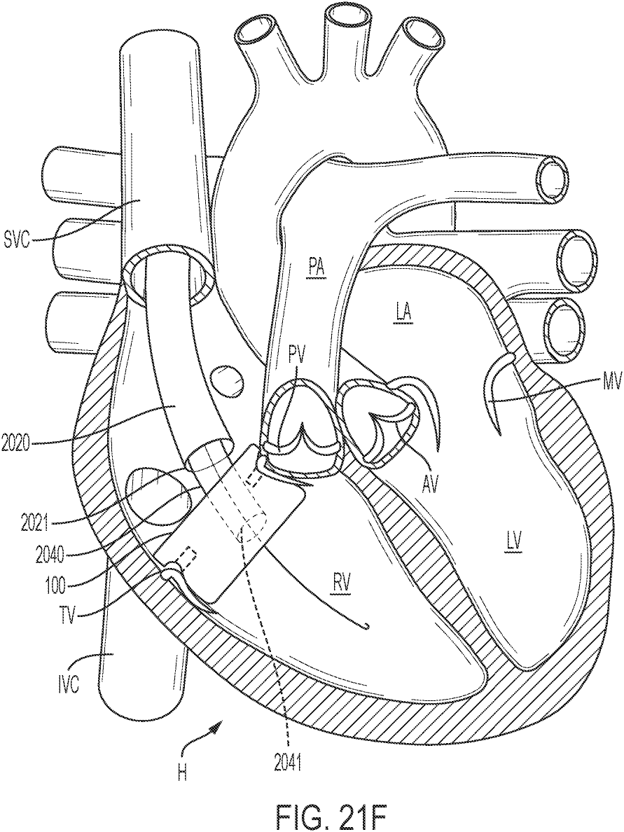

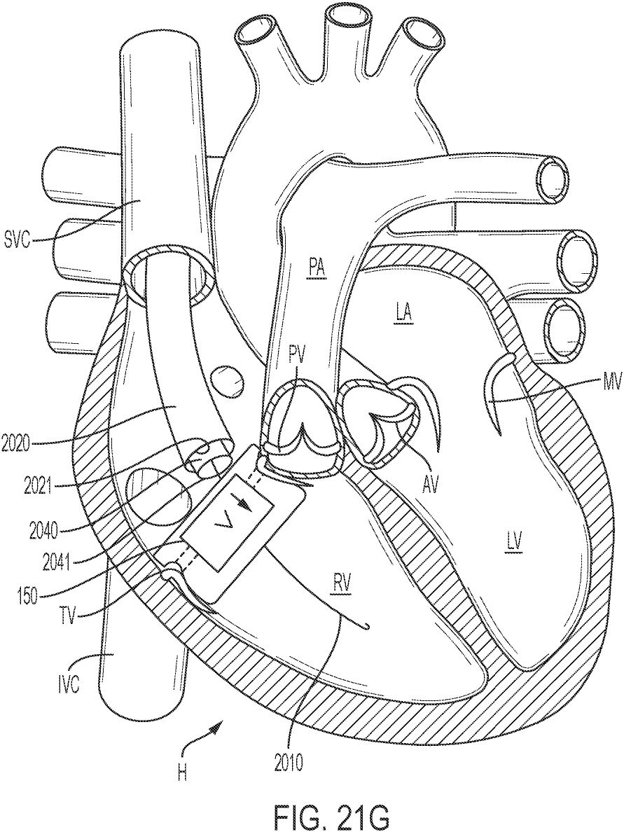

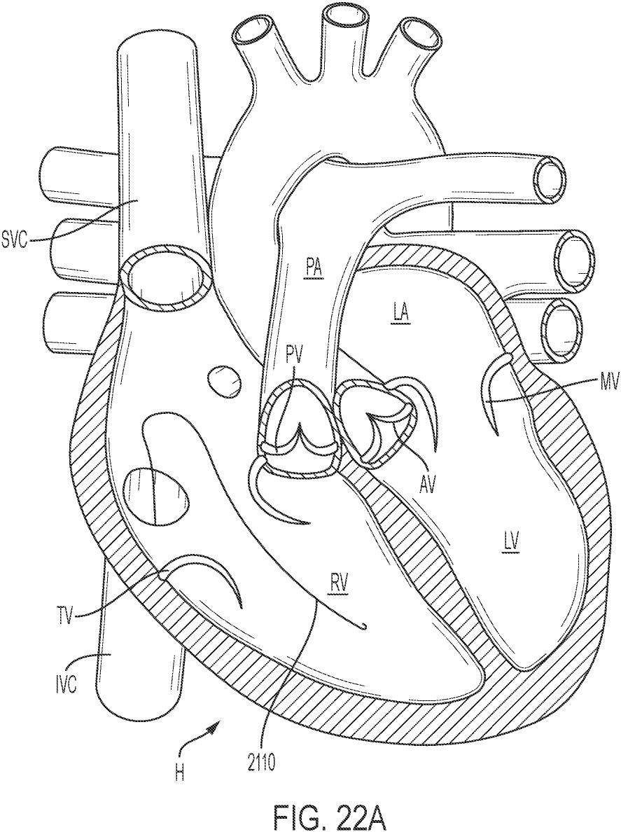

FIGS. 4A - 4D schematically illustrate an exemplary deployment of the docking station 100 and valve 150 in the circulatory system. Referring to FIG. 4A, the docking station 100 is in a compressed form/configuration and is introduced to a deployment site in the circulatory system. For example, the docking station 100 may be positioned at a deployment site (e.g., at the tricuspid valve TV annulus A) by a catheter (e.g., catheters 2000, 2100 as schematically shown in FIGS. 21A - 21G and 22A - 22E). Referring to FIG. 4B, the docking station 100 is expanded in the circulatory system such that the sealing portion(s) 130 and the retaining portions 120 engage the inside surface IS of a portion of the circulatory system. Referring to FIG. 4C, after the docking station 100 is deployed, the valve 150 is in a compressed form and is introduced into the valve seat 140 of the docking station 100. Referring to FIG. 4D, the valve 150 is expanded in the docking station 100, such that the valve engages the valve seat 140. In the examples depicted herein, the docking station 100 is longer than the valve 150. However, in other embodiments the docking station 100 can be the same length or shorter than the length of the valve 150. Similarly, the valve seat 140 can be longer, shorter, or the same length as the length of the valve 150.

Referring to FIG. 4D, the valve 150 has expanded such that the valve seat 140 of the docking station 100 supports the valve. The exemplary valve 150 only needs to expand against the valve seat 140, rather than against the wider space within the portion of the circulatory system that the docking station 100 occupies. The positioning of the valve seat 140 at the narrowed waist portion 113 of the docking station 100 allows the valve 150 to operate within the expansion diameter range for which it is designed.

Referring to FIG. 3, when the heart H is in the diastolic phase, the valve 150 opens. Blood flows from the inferior vena cava IVC and the superior vena cava SVC into the right atrium RA, and from the right atrium RA through the docking station 100 and open valve 150 into the right ventricle RV, as indicated by arrows A1. In an exemplary embodiment, blood is prevented from flowing between the right atrium RA and the docking station 100 by the at least one sealing portion 130 (see FIGS. 4B and 4C) and blood is prevented from flowing between the docking station and the valve 150 by seating of the valve in the valve seat 140 of the docking station 100, against the sealing portion(s). In this example, blood is substantially only or only able to flow between the right ventricle RV and the right atrium RA when the heart is in the diastolic phase (i.e., through the open valve 150).

When the heart is in the systolic phase, the valve 150 closes. Blood is prevented from flowing from the right ventricle RV into the right atrium RA by the valve 150 being closed, by the at least one sealing portion 130 between the docking station 100 and the interior surface IS of the circulatory system, and by seating of the valve in the valve seat 140 of the docking station 100, against the sealing portion(s).

In one exemplary embodiment, the docking station 100 acts as an isolator that prevents or substantially prevents radial outward forces of the valve 150 from being transferred to the inner surface IS of the circulatory system (e.g., the right ventricle RV, the right atrium RA, and the native tricuspid valve TV annulus A). In one embodiment, the docking station 100 includes a valve seat 140 which is not expanded radially outwardly or is not substantially expanded radially outward by the radially outward force of the THV or valve 150 (e.g., the diameter of the valve seat is not increased or is increased by less than 4 mm by the force of the THV), and retaining portions 120 and sealing portions 130 which impart only relatively small radially outward forces on the inner surface IS of the circulatory system (as compared to the radially outward force applied to the valve seat 140 by the valve 150).

Having a valve seat 140 that is stiffer or less radially expansive than the outer portions of the docking station (e.g., retaining portions 120 and sealing portions 130), as in the various docking stations described herein, provides many benefits, including allowing a THV/valve 150 to be implanted in vasculature or tissue of varying strengths, sizes, and shapes. The outer portions of the docking station can better conform to the anatomy (e.g., vasculature, tissue, heart, etc.) without putting too much pressure on the anatomy, while the THV/valve 150 can be firmly and securely implanted in the valve seat 140 with forces that will prevent or mitigate the risk of migration or slipping.

The docking station 100 can have any combination of one or more than one different types of valve seats 140 and sealing portions 130. In one exemplary embodiment, the valve seat 140 is a separate component that is attached to the body 110 of the docking station 100 and the sealing portion 130 is integrally formed with the body of the docking station. In another exemplary embodiment, the valve seat 140 is a separate component that is attached to the body 110 of the docking station 100 and the sealing portion 130 is a separate component that is attached to the body of the docking station. In another exemplary embodiment, the valve seat 140 is integrally formed with the body 110 of the docking station 100 and the sealing portion 130 is integrally formed with the body of the docking station. In still another exemplary embodiment, the valve seat 140 is integrally formed with the body 110 of the docking station 100 and the sealing portion is a separate component that is attached to the body of the docking station.

The one or more sealing portions 130, the valve seat 140, and the one or more retaining portions 120 can take a wide variety of different forms or combinations of forms. In many of the exemplary embodiments described herein, the docking station body 110 includes an expandable frame that provides the shape of the sealing portion(s) 130, the valve seat 140, and the retaining portion(s) 120. As described in greater detail below, the sealing portion(s) 130 of the docking station body 110 may include one or more impermeable materials (e.g., fabric, foam, and/or biocompatible tissue) secured to the expandable frame to effect a seal between the docking station body and the internal surface IS at the sealing portion(s), and a seal between the docking station body and the valve 150 at the valve seat 140. The sealing materials of the sealing portion(s) 130 may be integral to or in sealing engagement with each other.

The inner surfaces of the circulatory system, such as the inner surfaces of the right atrium RA and right ventricle RV adjacent to the tricuspid valve TV, can vary in cross-section size and/or shape along its length. In an exemplary embodiment, the docking station is configured to expand radially outwardly to varying degrees along its length to conform to shape of the inner surface. In one exemplary embodiment, the docking station 100 is configured such that the sealing portion(s) 130 and/or the retaining portion(s) 120 engage the internal surface IS, even though the surface contours vary significantly along the length of the docking station deployment site. The docking station can be made from a very resilient or compliant material to accommodate large variations in the anatomy.

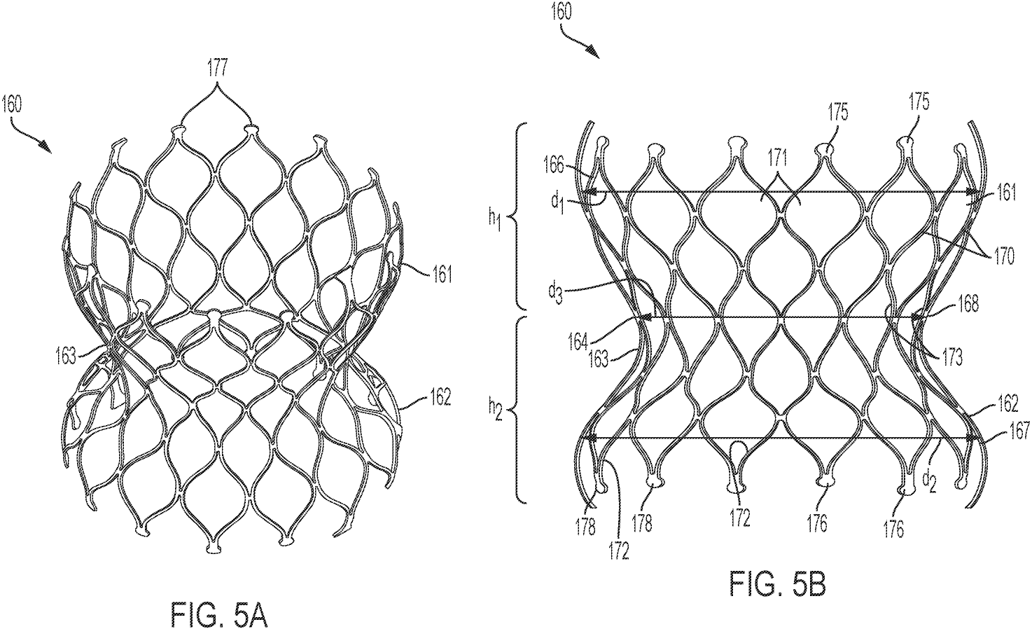

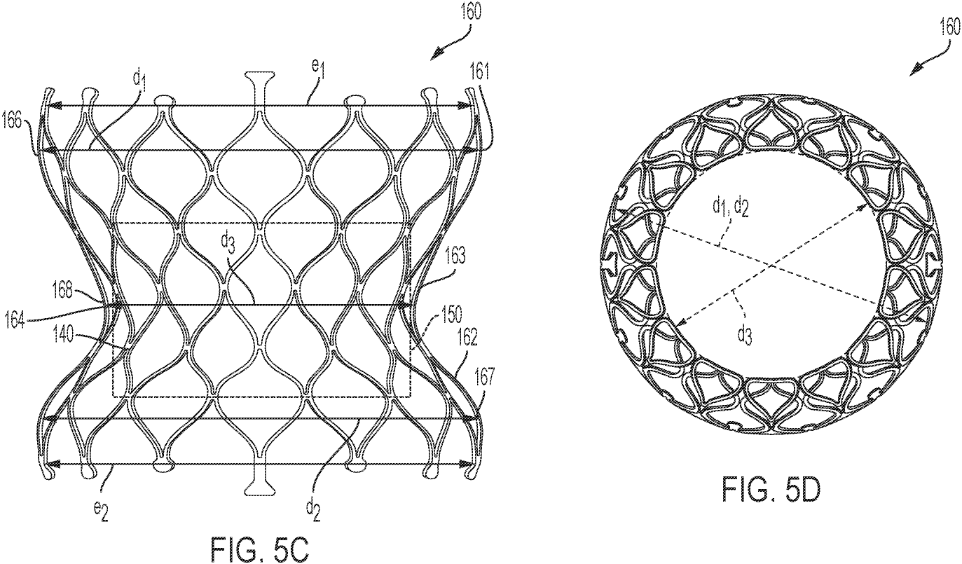

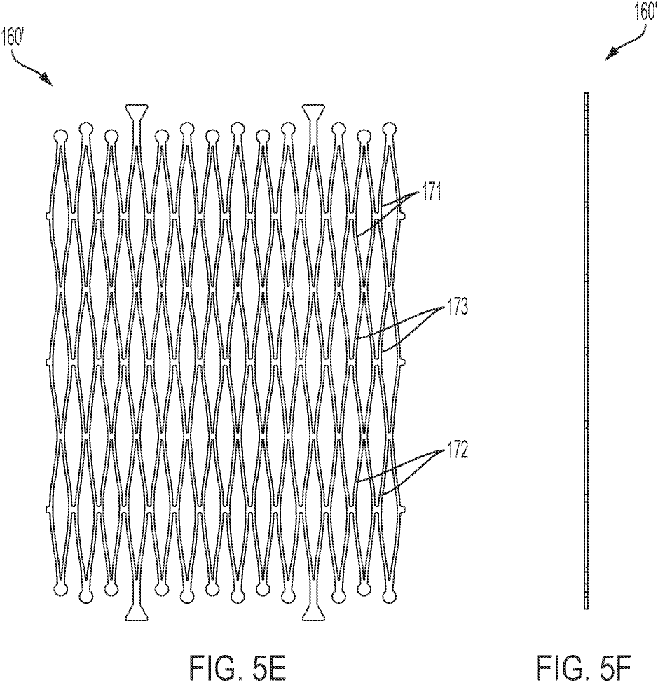

The expandable frame can take a wide variety of different forms. FIGS. 5A - 5D illustrate an exemplary expandable frame 160 having relatively wider first (e.g., inflow) and second (e.g., outflow) end portions 161, 162, and a relatively narrower central waist portion 163 that forms the valve seat between the end portions 161, 162. In one exemplary embodiment, the proximal end of the end portion 161 and/or the distal end of the end portion 162 extends radially inward. This radial inward extension of the end portion 161 and/or end portion 162 can prevent the proximal end of the end portion 161 and/or the distal end of the end portion 162 from contacting the vasculature.

In many of the exemplary embodiments described and illustrated in the present disclosure, the expandable frame is a wide stent including a plurality of struts that form an expandable lattice structure defining an array of cells. In the exemplary embodiment of FIGS. 5A - 5D, the expandable frame 160 has a plurality of flexible struts 170 forming a generally hourglass-shaped lattice structure defining one or more rows of distal or first end cells 171 that form the first (e.g., inflow) end portion 161, one or more rows of proximal or second end cells 172 that form the second (e.g., outflow) end portion 162, and one or more rows of central cells 173 that form the narrower waist portion 163 adapted to define the valve seat (as described in greater detail below). The cells 171, 172, 173 may be formed in a variety of shapes-in the illustrated example, the cells are substantially diamond-shaped, and longer in an axial direction than a lateral direction, for example, to allow for a greater range of expansion and contraction of the expandable frame 160. While the illustrated embodiment includes a single row of cells 171, 172, 173 in each of the first end portion 161, the second end portion 162, and the waist portion 163, in other embodiments, one or more of the first end portion, the second end portion, and the waist portion may include more than one row of cells. Additionally, each row of cells may include any suitable number of cells (e.g., 4 to 30 cells per row, such as 8 to 24 cells per row, such as 12 to 18 cells per row), and may include different numbers of cells in two or more of the rows. In the illustrated exemplary embodiment, the expandable frame 160 includes fourteen first end cells 171, fourteen second end cells 172, and fourteen central cells 173. In some applications, a greater number of cells and corresponding apices in the frame end portions (e.g., 12, 14, or more) may be used, for example, to provide for improved tissue contact and/or to maintain low loading forces when compressed or crimped in a delivery catheter.

As shown, the apices 175, 176 of the struts 170 may include enlarged foot portions 177, 178, which may, for example be configured for engagement with a frame deployment mechanism, such as, for example, a catheter. A variety of suitable catheters and other such deployment mechanisms may be used. For example, the exemplary docking stations and frames described herein may be adapted to be deployed using catheter systems described in the following references, the entire disclosures of each of which is incorporated herein by reference: U.S. Pat. Application Publication No. 2019/0000615 and U.S. Pat. No. 10,363,130.

As described below, the deployed valve 150 is expanded in the waist portion 163 of the expandable frame 160, which forms the valve seat 140. The expandable lattice can be made from individual wires or can be cut from a sheet and then rolled or otherwise formed into the shape of the expandable frame. FIGS. 5E and 5F illustrate a lattice sheet 160′, cut or otherwise formed (e.g., by 3D printing) from a desired material to form the distal, proximal, and central cells 171, 172, 173. The lattice sheet 160′ may be rolled or otherwise formed into the desired shape of the expandable frame 160.

The expandable frame 160 can be made from a highly flexible metal, metal alloy, or polymer. Examples of metals and metal alloys that can be used include, but are not limited to, nitinol and other shape memory alloys, elgiloy, and stainless steel, but other metals and highly resilient or compliant non-metal materials can be used to make the expandable frame 160. These materials can allow the frame to be compressed to a small size (e.g., within a catheter), and then when the compression force is released (e.g., the frame is extended from the catheter), the frame may self-expand back to its pre-compressed diameter. Alternatively, the compressed frame may be forcibly expanded, for example, by inflation of a device positioned inside the frame.

The first end portion 161, the second end portion 162, and the narrowed waist portion 163 may be provided in a variety of sizes and shapes to accommodate the intended deployment site and/or the seated valve. In the illustrated embodiment of FIGS. 5A - 5D, the first and second end portions 161, 162 have substantially circular outer radial portions 166, 167 having substantially equal major lateral dimensions (e.g., outer diameters) d₁, d₂ (e.g., between about 48 mm and about 50 mm) and the waist portion 163 has a substantially circular inner radial portion 168 having a major lateral dimension (e.g., outer diameter) d₃ (e.g., about 27 mm) significantly smaller than the dimensions d₁, d₂ and defining a valve seat 140 sized to accommodate the expanded valve 150. As shown, the expandable frame 160 may be substantially longitudinally symmetrical or substantially symmetrical about a lateral plane bisecting an axial midpoint of the frame (e.g., at a midpoint 164 of the waist portion 163), such that a first axial length h₁ (e.g., about 17.5 mm) of the first end portion (i.e., distance from the axial midpoint 164 of the waist portion 163 to the frame strut apices 175 of the first end portion 161) is substantially equal to a second axial length h₂ of the second end portion (i.e., distance from the axial midpoint 164 of the waist portion 163 to the frame strut apices 176 of the second end portion 162).

The exemplary frames described herein may be provided with a variety of suitable axial lengths, for example, to accommodate different sizes and types of deployment sites, including, for example, tricuspid regions having different shapes and dimensions. As one example, an expandable frame may have an axial length or height between about 31 mm and about 39 mm, or about 35 mm. As another example, an expandable frame may have an axial length or height between about 39 mm and about 45 mm, or about 42 mm.

As shown, the geometry of the frame, as shown in FIGS. 5A - 5D and described above, may produce a substantially longitudinally and circumferentially symmetrical hourglass shape when the frame 160 is in an expanded, unconstrained condition, with the first and second end portions 161, 162 defining convex retaining portions 120 and the waist portion 163 defining a concave valve seat 140. As shown, the distal and proximal apices 175, 176 of the frame struts 170 may be contoured radially inward, for example, to limit, minimize, or prevent tissue contact by the apices at the deployment site upon implantation of the docking station 100, and/or to ensure retaining engagement by the frame struts along the extended convex surface of the frame struts. In the exemplary embodiment of FIGS. 5A - 5D, these inward contoured apices 175, 176 produce substantially equal circular open ends having opening major lateral dimensions (e.g., diameters) e₁, e₂ (e.g., about 48.4 mm) at least slightly smaller than the major lateral dimensions d₁, d₂ of the first and second end portions 161, 162.

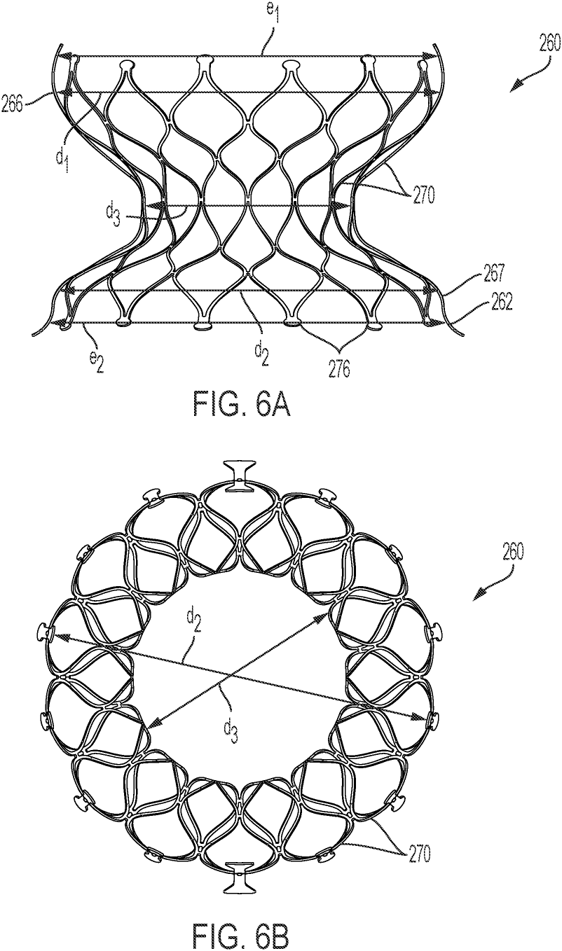

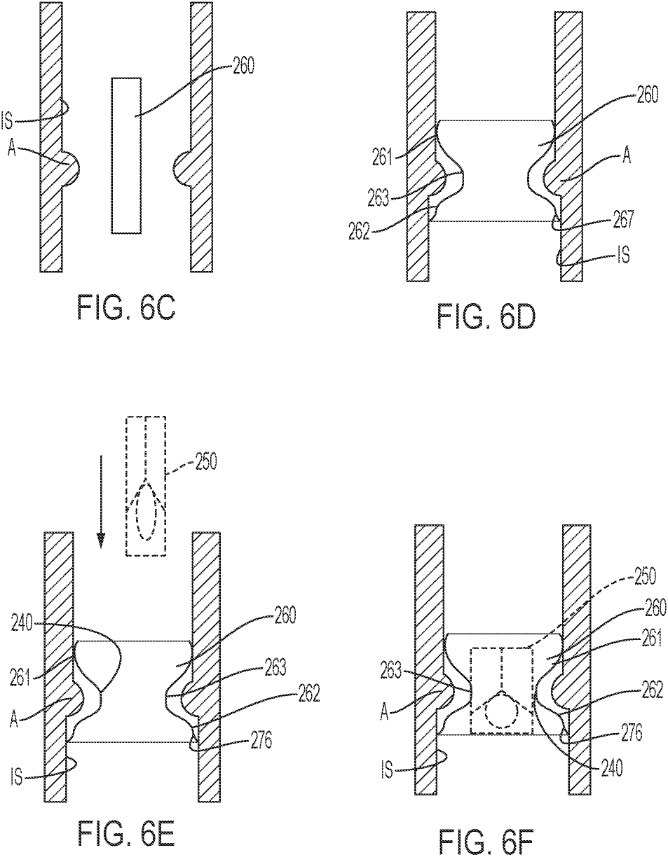

According to another exemplary aspect of the present disclosure, in other embodiments, a frame may include a plurality of flexible struts having distal and/or proximal apices that are contoured radially outward, for example, to provide reinforced anchor points to secure the expandable frame to circulatory tissue at the deployment site. FIGS. 6A and 6B illustrate an exemplary expandable frame 260 formed from a plurality of flexible struts 270 with a radially inward contoured first (e.g., inflow) end portion 261, similar to the expandable frame 160 of FIGS. 5A - 5D, but having a second (e.g., outflow) end portion 262 having radially outward contoured or flared proximal apices 276. In the exemplary embodiment of FIGS. 6A and 6B, the outward contoured apices 276 produce a circular open end having a major lateral dimension (e.g., opening diameter) e₂ (e.g., about 52.7 mm) at least slightly larger than the outer diameters d₁, d₂ (e.g., about 50 mm) of the outer radial portions 266, 267. In an exemplary application, as shown in FIGS. 6C - 6F, the expandable frame 260 is deployed at the vasculature, such as at the tricuspid valve TV, with the outward contoured proximal apices 276 anchoring the frame to the vasculature, such as the right ventricle or the right atrium (depending on the orientation of the docking station). Referring to FIG. 6E, after the expandable frame 260 is deployed, the valve 250 is in a compressed form and is introduced into the valve seat 240 of the expandable frame 260. Referring to FIG. 6F, the valve 250 is expanded in the expandable frame 260, such that the valve engages the valve seat 240.

Referring back to FIGS. 5A - 5D, the concave structure of the expandable frame waist portion 163 may be configured to provide clearance for circulatory tissue at the deployment site (e.g., the tricuspid valve TC annulus A) and to resist or absorb radially outward forces of the deployed and expanded valve, thereby securing the valve within the docking station 100 at the valve seat 140. The diameter d₃ of the inner radial portion of the waist portion 163, in an unconstrained state, may be sized to be at least slightly smaller (e.g., about 2 mm smaller) than an outer diameter of the expanded valve 150, such that the waist portion 163 is at least slightly expanded by the radially outward forces of the deployed and expanded valve to secure the valve to the valve seat. By limiting this expansion of the waist portion 163 and maintaining a generally narrowed (e.g., concave) shape of the waist portion 163, the relatively high radially outward forces from the expanded valve are isolated from the vasculature of the circulatory system.

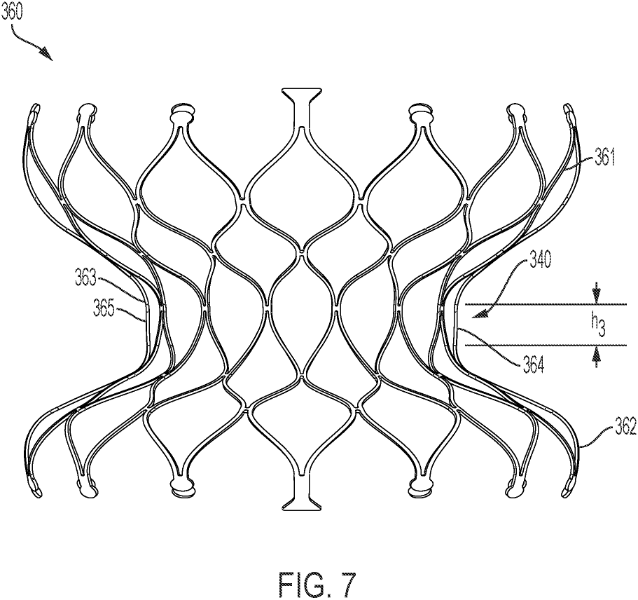

While the concave waist portion 163 may have a continuous arcuate profile, as shown in FIGS. 5A - 5D, in other embodiments, at least a portion of the waist portion may have a flattened or axial (e.g., tubular or cylindrical) profile, for example, to provide an axially elongated valve seat for uniformly engaging and sealing against the expanded valve over an extended axial surface. FIG. 7 illustrates an expandable frame 360 having wider first and second end portions 361, 362 and a narrower waist portion 363 including a flat section 365 extending over an axial length or height h₃ (e.g., about 3 mm) defining a valve seat 340 sized to accommodate a seating portion of an expanded valve. The flat section 365 may (but need not) extend over the axial midpoint 364 of the waist portion 363, and may (but need not) be centered on the axial midpoint.

Referring back to FIGS. 5A - 5D, the convex structure of the retaining portions 120 may be configured to apply, to the internal surface IS at the deployment site, a radially outward retaining force that is substantially smaller than the radially outward seating force applied by the valve to the valve seat 140. For example, the radially outward retaining force can be less than 75% of the radially outward force applied by the valve, such as less than 50% of the radially outward force applied by the valve, such as less than 25% of the radially outward force applied by the valve, such as less than 10% of the radially outward force applied by the valve. As one example, when using a valve (e.g., a 29 mm size Sapien 3 prosthetic valve) that typically applies a radially outward force of about 42 Newtons, the retaining portions 120 may be configured to apply a radially outward force (chronic outward force, or COF) of between 10 and 35 Newtons, such as between 15 and 30 Newtons, such as between 23 and 27 Newtons, such as 25 Newtons. The convex contours of the retaining portion(s) may be configured to apply this retaining force over an extended longitudinal surface of the docking station. For example, the retaining portion(s) can be configured to apply this retaining force over 20-80% of the longitudinal surface of the docking station, such as at least 30-70% of the longitudinal surface of the docking station, such as 40-60% of the longitudinal surface of the docking station.

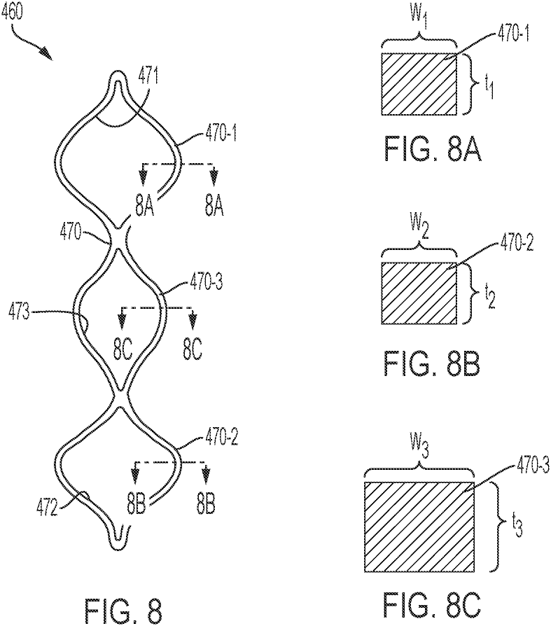

As discussed above, the generally convex shape of the retaining portions 120 may be configured to apply a relatively low retaining force to the internal surface IS at the deployment site (e.g., to be atraumatic to the deployment site), and the generally concave shape of the waist portion 163 (defining the valve seat 140) may be configured to apply a relatively large retaining force to the expanded valve. According to another exemplary aspect of the present disclosure, the frame struts 170 may be configured to vary in circumferential width and/or radial thickness to provide increased or decreased flexibility and/or increased or decreased radial forces for desired engagement between the retaining portions 120 and the internal surface IS and between the valve seat 140 and the valve. In one such embodiment, as shown in FIGS. 8 and 8A -8C, the struts 470 of an expandable frame 460 may have distal first end portions 470-1 (defining the distal or first end cells 471) having a first cross-sectional area, second end or proximal portions 470-2 (defining the proximal or second end cells 472) having a second cross-sectional area, and central portions 470-3 (defining the central cells 473) having a third cross-sectional area. In the illustrated example, the third cross-sectional area of the central strut portions 470-3 is greater than the first and second cross-sectional areas of the first and second end strut portions 470-1, 470-2 (which, may, but need not, be substantially the same). The greater cross-sectional area of the central strut portions 470-3 may provide for reduced flexibility (e.g., to isolate the valve seating waist portion from the deployment site) and increased radial force (e.g., to securely retain the seated valve) at the central portion of the frame 460, while the smaller cross-sectional area of the first and second end strut portions 470-1, 470-2 may provide for increased flexibility (e.g., to conform to the internal surface IS contours at the deployment site) and reduced but sufficient radial force (e.g., to minimize or prevent tissue damage by the retaining portions) at the proximal and distal end portions, for example, to maintain a chronic outward force (COF) of at least about 25 Newtons for sufficient anchoring of the frame 460 at the triscuspid valve TV annulus while maintaining flexibility for compliance with the contours of the tissue at the deployment site. As shown, the central strut portions 470-3 may have a greater radial thickness t₃ than a thickness t₁ and/or t₂ of the distal and/or proximal strut portions 470-1, 470-2 and/or a greater circumferential width w₃ than a width w₁ and/or w₂ of the distal and/or proximal strut portions 470-1, 470-2. For example, the radial thickness t₃ can be 125% to 300% of the thickness t₁ and/or t₂, such as 150% to 250% of the thickness t₁ and/or t₂, such as 175% to 225% of the thickness t₁ and/or t₂. The circumferential width w₃ can be 125% to 300% of the width w₁ and/or w₂, such as 150% to 250% of the width w₁ and/or w₂, such as 175% to 225% of the width w₁ and/or w₂.

Other arrangements may additionally or alternatively be used to provide a stiffer/less flexible and increased radial force applying waist portion. For example, as schematically shown in FIG. 4B, the docking station body 110 may include a band 119 extending about the waist portion 113, assembled with or integral to the waist portion to form an unexpandable or substantially unexpandable valve seat 140. The band 119 stiffens the waist portion and, once the docking station is deployed and expanded, makes the waist/valve seat relatively unexpandable in its deployed configuration. The unexpandable or substantially unexpandable valve seat 140 can prevent the radially outward force of the valve 150 from being transferred to the inside surface IS of the circulatory system. However in another exemplary embodiment, the waist/valve seat of the deployed docking station can optionally expand slightly in an elastic fashion when the valve 150 is deployed against it. This optional elastic expansion of the waist portion 113 can put pressure on the valve 150 to help hold the valve in place within the docking station.

The band 119 can take a wide variety of different forms and can be made from a wide variety of different materials. The band 119 can be made of PET, one or more sutures, fabric, metal, polymer, a biocompatible tape, or other relatively unexpandable materials known in the art that are sufficient to maintain the shape of the valve seat 140 and hold the valve 150 in place. The band can extend about the exterior of the frame, or can be an integral part of it, such as when fabric or another material is interwoven into or through cells of the stent. The band can be a variety of widths, lengths, and thicknesses. The valve 150, when docked within the docking station, can optionally expand around either side of the valve seat slightly. This aspect, sometimes referred to as a “dogbone” (e.g., because of the shape it forms around the valve seat or band), can also help hold the valve in place.

In other embodiments, flexibility of the expandable frame along its length may be varied by varying the shape and/or size of the first end, second end, and central cells of the frame. For example, referring to the cells 471, 472, 473 of FIG. 8, the first and second end cells 471, 472 may be relatively longer (e.g., an axial length or height of about 12 mm) to provide for increased flexibility of the end portions, and the central cells 473 may be relatively shorter (e.g., an axial length or height of about 10.5 mm) to provide for reduced flexibility or increased rigidity at the waist portion of the frame.

While the docking station arrangements described herein may be used at a variety of deployment sites, in one exemplary application described herein, a docking station (e.g., any of the exemplary docking stations described herein) may be deployed at the native tricuspid valve TV, with an enlarged first inflow end portion sized and configured to be retained in the right atrium RA distal to the tricuspid valve TV annulus, an enlarged second outflow end portion sized and configured to be retained in the right ventricle RV proximal to the native tricuspid valve TV annulus, and a narrowed central portion or waist portion 113 sized and configured to align with and accommodate the native tricuspid valve TV.

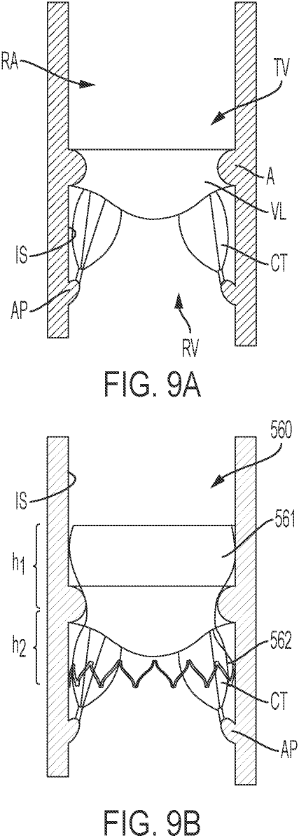

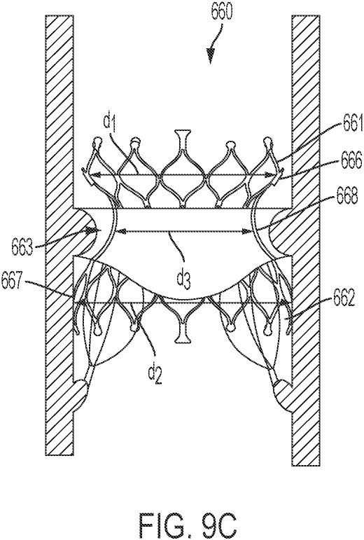

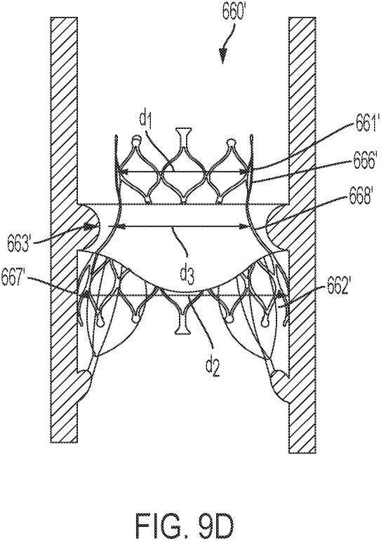

Several characteristics of the tricuspid valve TV and the portions of the right atrium RA and right ventricle RV adjacent to the tricuspid valve can present challenges for implanting a THV at the tricuspid valve annulus, including, for example, the enlarged and non-calcified nature of the tricuspid valve annulus, the contours of the right atrium RA and right ventricle RV, the proximity of the tricuspid valve to the pulmonary valve PV in the right ventricle RV and to the inferior vena cava IVC in the right atrium RA, and the presence of the chordae tendineae extending from the native tricuspid valve TV leaflets and anchored to the walls of the right ventricle RV at anchor points AP. FIG. 9A schematically illustrates the tricuspid valve TV region of the heart, including a tricuspid valve annulus A extending radially inward from an internal surface IS, native valve leaflets VL extending radially inward from the annulus, and chordae tendineae CT extending from the valve leaflets to the internal surface of the right ventricle RV.

According to one or more exemplary aspects of the present disclosure, the geometry of the expandable frame may be configured or adapted to better function at the intended deployment site, such as, for example, at the tricuspid valve TV annulus. For example, rather than being longitudinally symmetrical about an axial midpoint of the frame (as described above), the frame may be longitudinally asymmetrical, with one of the first and second end portions having a greater axial length or height, and the other of the first and second end portions having a smaller axial length or height. In the exemplary embodiment of FIG. 9B, the expandable frame 560 includes a shorter outflow end portion 562 (e.g., an axial length h₂ of about 15 mm), for example, to minimize or prevent damage to the chordae tendineae CT in the right ventricle RV (e.g., by eliminating or minimizing frame contact with the chordae tendineae anchor points AP), and a longer inflow end portion 561 (e.g., an axial length h₁ of about 20 mm), for example, to apply retention forces over an increased interior surface of the right atrium RA. In other exemplary applications, an expandable frame may include a shorter inflow end portion and a longer outflow end portion.

As another example, rather than having first and second end portions with outer radial portions that are substantially equal in size (as described above), one of the first and second end portions may have an outer radial portion that is smaller than the outer radial portion of the other end portion, but still larger than an inner radial portion of the waist portion. In one such example, as shown in FIG. 9C, an expandable frame 660 includes a second (e.g., outflow) end portion 662 having an outer radial portion 667 with an outer diameter d₂ larger than an outer diameter d₁ of an outer radial portion 666 of a first (e.g., inflow) end portion 661, for example to anchor the docking station primarily or entirely to the right ventricle when the docking station is installed at the tricuspid valve annulus. This smaller outer diameter d₁ is larger than the outer diameter d₃ of the inner radial portion 668 of the waist portion 663 and may, but need not, still be expandable to engage the inner surface of the vasculature. In other applications, an expandable frame may have a first end outer radial portion larger than the second end outer radial portion, or the first and second end flange portion sizes may differ to varying degrees.

In another exemplary embodiment, as shown in FIG. 9D, an expandable frame 660′ may include a first (e.g., inflow) end portion 661′ that extends substantially or entirely axially from the waist portion 663′, such that the outer diameter d₁ of the outer radial portion 666′ of the first end portion is substantially the same as the diameter d₃ of the inner radial portion 668′ of the waist portion, and significantly smaller than the outer diameter d₂ of the outer radial portion 667′ of the second end portion 662′. In such an arrangement, the first end portion 661′ may not engage the inner surface of the vasculature, with the frame 660′ instead relying solely on engagement between the second (e.g., outflow) end portion 662′ and the inner surface for retention of the frame at the deployment site.

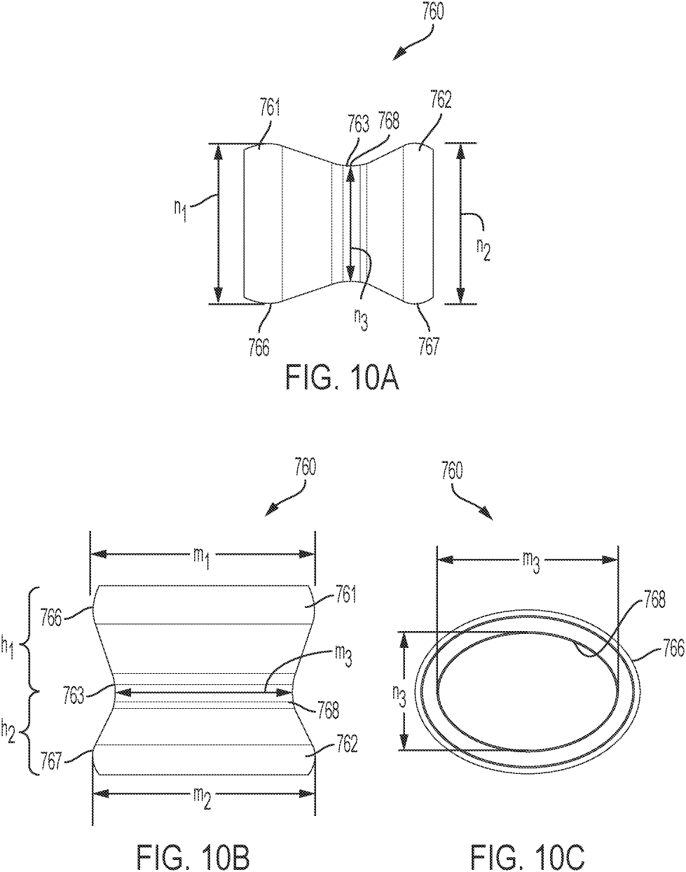

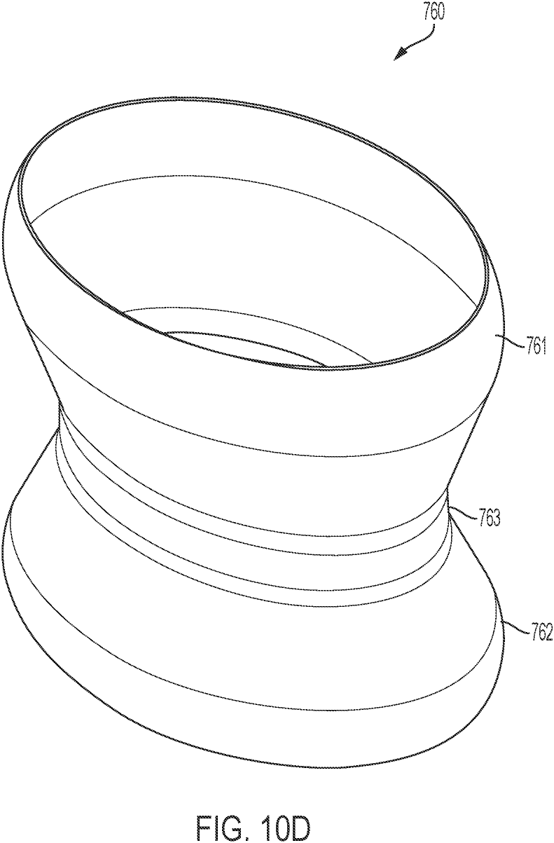

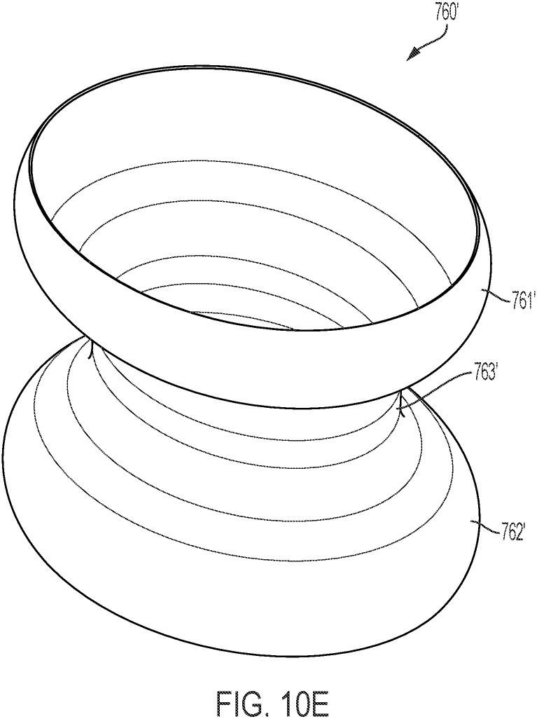

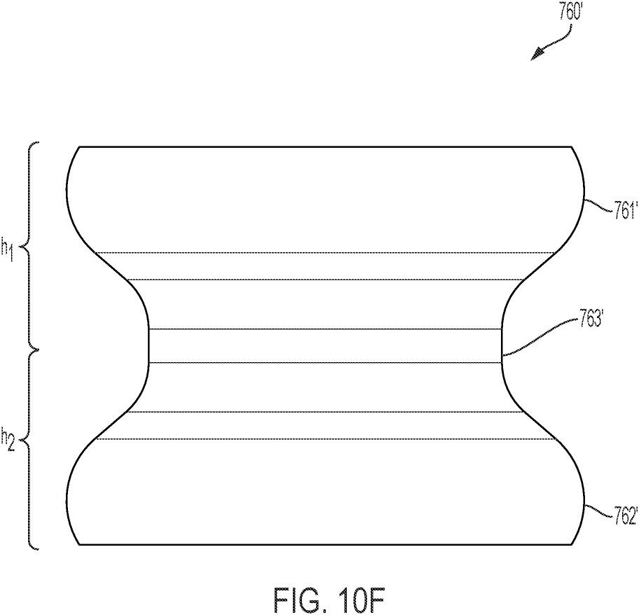

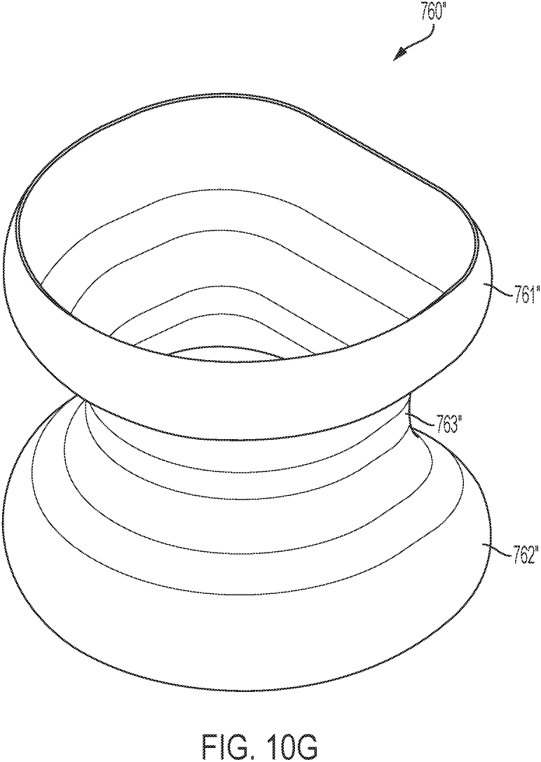

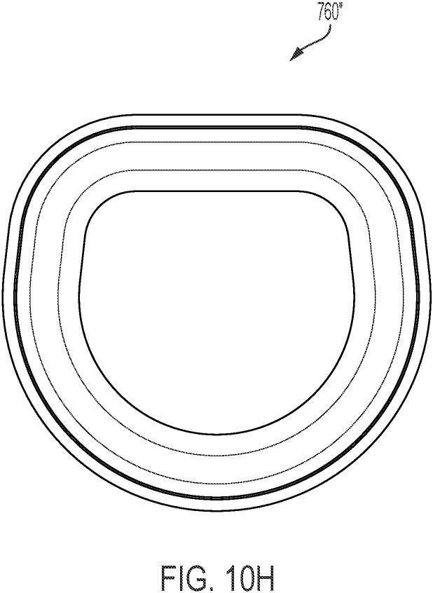

In some exemplary embodiments, rather than having first and second end portions 161, 162 and a waist portion 163 that are substantially circular in cross-section (as shown in FIGS. 5A - 5D and described above), the first end portion, the second end portion, and/or the waist portion may have a non-circular cross-section selected to better accommodate the cross-sectional shape of the internal surface IS at the deployment site, such as, for example, elliptical (of varying major-to-minor diameter ratios), semicircular, D-shaped, a rounded D-shape, generally wedge-shaped, generally trapezoidal shaped, and/or a combination of any of these shapes. In one such example, as shown in FIGS. 10A - 10D, an expandable frame 760 includes a first (e.g., inflow) end portion 761, a second (e.g., outflow) end portion 762 and a waist portion 763 each having an elliptical cross section, for example, to better conform to the oblong cross-sectional anatomy at and near the tricuspid valve TV for a docking station for a prosthetic tricuspid valve. In one such exemplary embodiment, the first end portion 761 and the second end portion 762 have substantially elliptical outer radial portions 766, 767 having substantially equal major diameters m₁, m₂ and/or substantially equal minor diameters n₁, n₂, and the waist portion 763 has a substantially elliptical inner radial portion 768 having a smaller major diameter m₃ and a smaller minor diameter n₃ than the outer radial portions. The frame 760 includes a includes a shorter outflow end portion 762 (e.g., an axial length h₂ of about 15 mm), for example, to minimize or prevent damage to the chordae tendineae CT in the right ventricle RV (e.g., by eliminating or minimizing frame contact with the chordae tendineae anchor points AP), and a longer inflow end portion 761 (e.g., an axial length h₁ of about 20 mm), for example, to apply retention forces over an increased interior surface of the right atrium. The schematically shown frame 760 may include cell-defining struts similar to the exemplary frames described above.

In another exemplary embodiment, as shown in FIGS. 10E - 10F, an expandable frame 760′ has an elliptical cross-section and a flat waist portion 763′, similar to the expandable frame 760 of FIGS. 10A - 10D, but with more bulbous, rounded end portions 761′, 762′ having substantially equal axial lengths h₁, h₂.

In another exemplary embodiment, as shown in FIGS. 10G - 10H, an expandable frame 760″ has a substantially D-shaped cross-section at the end portions 761″, 762″ and waist portion 763″, for example, to better accommodate the cross-sectional shape of the internal surface at the deployment site.

According to one or more exemplary aspects of the present disclosure, an expandable frame may include a wide variety of end portion axial lengths, end portion and waist portion cross-sectional sizes, and/or end portion and waist portion cross-sectional shapes, for example, to accommodate a variety of deployment sites in the circulatory system of a variety of human subjects.

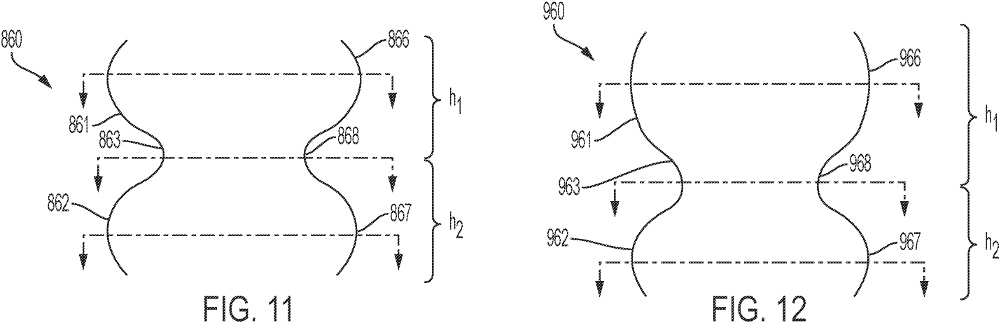

FIG. 11 generally illustrates a side view of an exemplary expandable frame 860 having first and second end portions 861, 862 of substantially equal axial length or height h₁, h₂ and having outer radial portions 866, 867 that are substantially the same size, and a narrowed waist portion 863 having an inner radial portion 868 smaller in size that the outer radial portions 866, 867. FIG. 12 generally illustrates a side view of an exemplary expandable frame 960 having a first (e.g., inflow) end portion 961 with a first (e.g., greater) axial length h₁ and a second (e.g., outflow) end portion 962 with a second (e.g., smaller) axial length h₂, with the first and second end portions 961, 962 having outer radial portions 966, 967 that are substantially the same size, and a narrowed waist portion 963 having an inner radial portion 968 smaller in size than the outer radial portions 966, 967. In other embodiments, the relative axial lengths h₁, h₂ may differ-for example, the axial length of the second end portion may be substantially equal to or greater than the axial length of the first end portion, or the axial lengths may differ to varying degrees. The exemplary frames 860, 960 may include cell-defining struts similar to those described above.

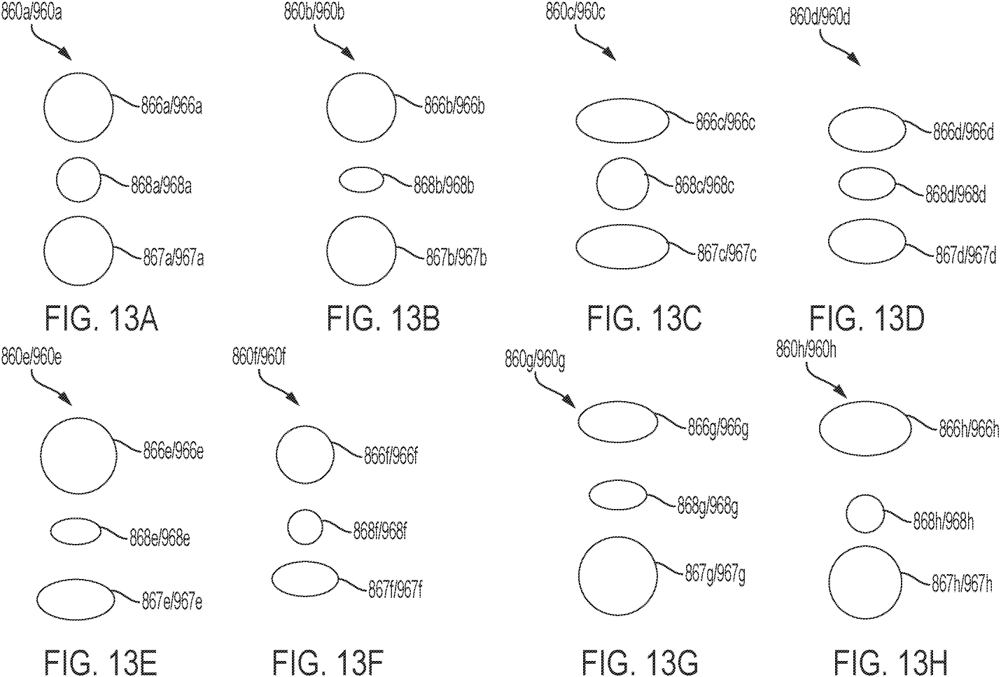

FIGS. 13A - 13H illustrate cross sectional views of outer radial portions 866a-h/966ah, 867a-h/967a-h and inner radial portions 868a-h/968a-h of various exemplary expandable frames 860a-h/960a-h corresponding to the expandable frames 860, 960 of FIGS. 11 and 12. For example, the expandable frame may include: circular outer radial portions 866a/966a, 867a/967a and a circular inner radial portion 868a/968a (FIG. 13A); circular outer radial portions 866b/966b, 867b/967b and an elliptical inner radial portion 868b/968b (FIG. 13B); elliptical outer radial portions 866c/966c, 867c/967c and a circular inner radial portion 868c/968c (FIG. 13C); elliptical outer radial portions 866d/966d, 867d/967d and an elliptical inner radial portion 868d/968d (FIG. 13D); a circular first outer radial portion 866e/966e, an elliptical second outer radial portion 867e/967e, and an elliptical inner radial portion 868e/968e (FIG. 13E); a circular first outer radial portion 866f/966f, an elliptical second outer radial portion 867f/967f, and a circular inner radial portion 868f/968f (FIG. 13F); an elliptical first outer radial portion 866g/966g, a circular second outer radial portion 867g/967g, and an elliptical inner radial portion 868g/968g (FIG. 13G); or an elliptical first outer radial portion 866h/966h, a circular second outer radial portion 867h/967h, and a circular inner radial portion 868h/968h (FIG. 13H). In other embodiments, the circular and/or elliptical cross-sectional shapes may be replaced with other suitable cross-sectional shapes, including, for example, D-shaped, rounded D-shaped, semicircular, generally wedge-shaped, and/or generally trapezoidal shaped.

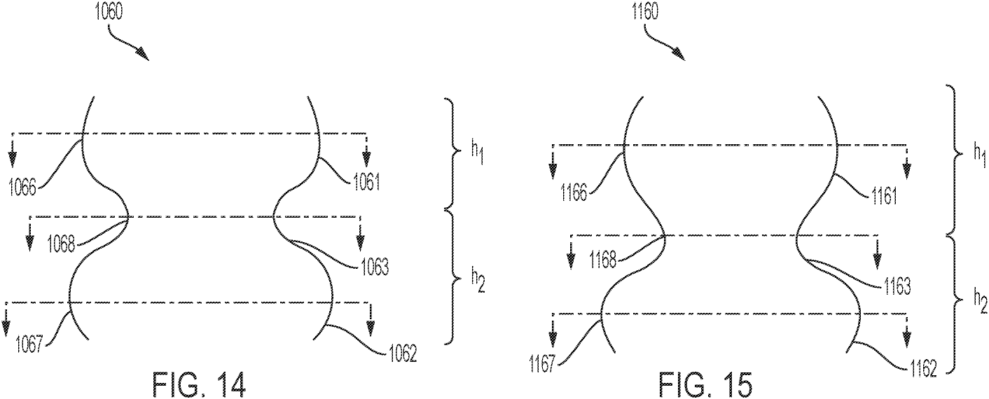

FIG. 14 generally illustrates a side view of an exemplary expandable frame 1060 having first and second end portions 1061, 1062 of substantially equal axial length h₁, h₂ and having an inflow end outer radial portion 1066 of a first size, an outflow end outer radial portion 1067 of a second size (e.g., larger than the first size, as shown), and a narrowed waist portion 1063 having an inner radial portion 1068 smaller in size than the outer radial portions 1066, 1067. In other embodiments (not shown) the second size of the outflow end outer radial portion may be smaller than the first size of the inflow end outer radial portion, or the outer radial portion sizes may differ to varying degrees. FIG. 15 generally illustrates a side view of an exemplary expandable frame 1160 having an inflow end portion 1161 with a first (e.g., greater) axial length h₁ and an outflow end portion 1162 with a second (e.g., smaller) axial length h₂, with an inflow end outer radial portion 1166 of a first size, an outflow end outer radial portion 1167 of a second size (e.g., larger than the first size, as shown), and a narrowed waist portion 1163 having an inner radial portion 1168 smaller in size that the outer radial portions 1166, 1167. In other embodiments, the relative axial lengths may differ-for example, the axial length of the outflow end portion may be greater than the axial length of the inflow end portion, or the axial lengths may differ to varying degrees. Additionally or alternatively, the second size of the outflow end outer radial portion may be smaller than the first size of the inflow end outer radial portion, or the outer radial portion sizes may differ to varying degrees. The exemplary frames 1060, 1160 may include cell-defining struts similar to those described above.

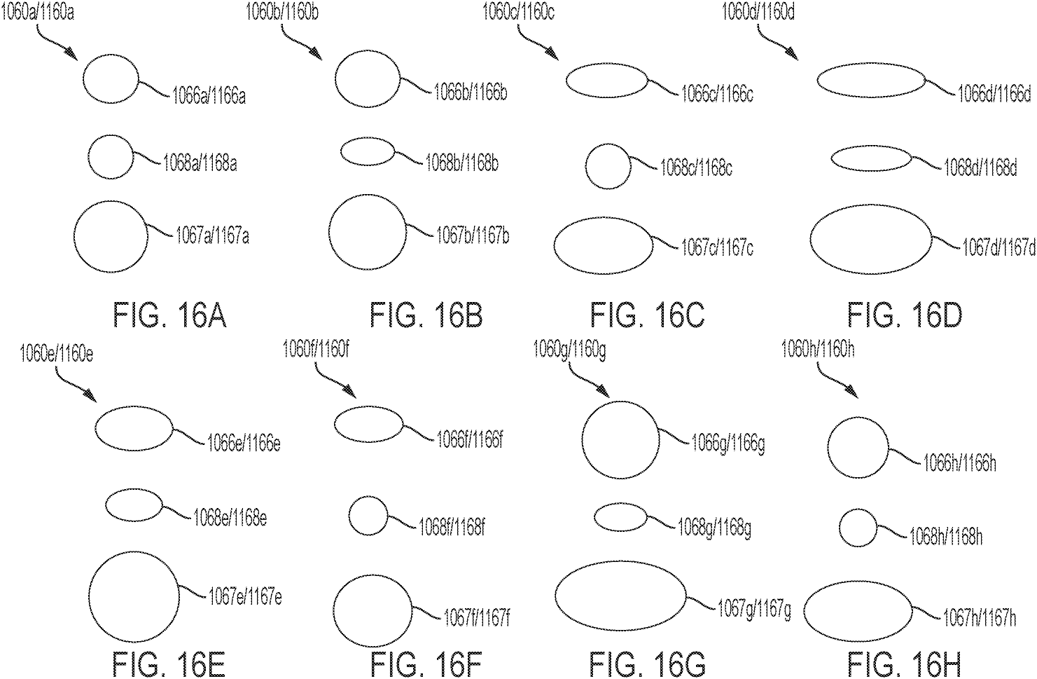

FIGS. 16A - 16H illustrate cross sectional views of outer radial portions 1066a-h/1166a-h, 1067a-h/1167a-h and inner radial portions 1068a-h/1168a-h of various exemplary expandable frames 1060a-h/1160a-h corresponding to the expandable frames 1060, 1160 of FIGS. 14 and 15. For example, the expandable frame may include: circular outer radial portions 1066a/1166a, 1067a/1167a and a circular inner radial portion 1068a/1168a (FIG. 16A); circular outer radial portions 1066b/1166b, 1067b/1167b and an elliptical inner radial portion 1068b/1168b (FIG. 16B); elliptical outer radial portions 1066c/1166c, 1067c/1167c and a circular inner radial portion 1068c/1168c (FIG. 16C); elliptical outer radial portions 1066d/1166d, 1067d/1167d and an elliptical inner radial portion 1068d/1168d (FIG. 16D); a circular outflow outer radial portion 1066e/1166e, an elliptical inflow outer radial portion 1067e/1167e, and an elliptical inner radial portion 1068e/1168e (FIG. 16E); a circular outflow outer radial portion 1066f/1166f, an elliptical inflow outer radial portion 1067f/1167f, and circular inner radial portion 1068f/1168f (FIG. 16F); an elliptical outflow outer radial portion 1066g/1166g, a circular inflow outer radial portion 1067g/1167g, and an elliptical inner radial portion 1068g/1168g (FIG. 16G); and an elliptical outflow outer radial portion 1066h/1166h, a circular inflow outer radial portion 1067h/1167h, and a circular inner radial portion 1068h/1168h (FIG. 16H). In other embodiments, the circular and/or elliptical cross-sectional shapes may be replaced with other suitable cross-sectional shapes, including, for example, “D” shaped, rounded “D” shapes, semicircular, generally wedge-shaped, shapes that mimic the shape of the native tricuspid valve, shapes that mimic the shape of the native mitral valve, a generally trapezoidal shaped and/or any combination of these shape or combinations of these shapes with other shapes.

The sealing portion(s) of a docking station, such as the exemplary embodiments described herein, can take a wide variety of different forms. Referring back to the schematically illustrated exemplary embodiment of FIGS. 4A - 4D, one or more impermeable coverings (e.g., a biocompatible fabric or foam) may be attached to a portion of the docking station body 110 to form the sealing portion 130, to provide a seal between the valve 150 and the internal surface IS at the deployment site. The sealing portion 130 can take any form the prevents the flow of blood from flowing around the outside surface of the valve 150 and through the docking station 100.

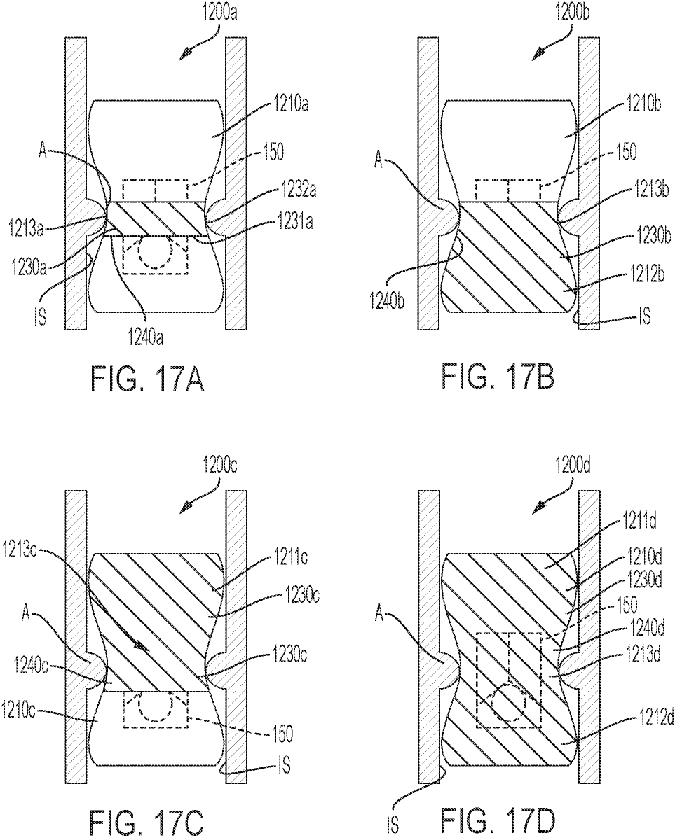

In some embodiments, a docking station may include a sealing portion axially aligned with the valve seat to provide a seal between the valve and the internal surface IS aligned with the waist portion of the docking station body. FIG. 17A schematically illustrates an exemplary docking station 1200a including a sealing portion 1230a attached to the docking station body 1210a and limited to the waist portion 1213a of the body, such that the sealing portion includes an inner seal portion 1231a that seals against the valve 150 (e.g., at the valve seat 1240a) and an outer seal portion 1232a that seals against the internal surface IS (e.g., at the annulus A) aligned with the waist portion 1213a.

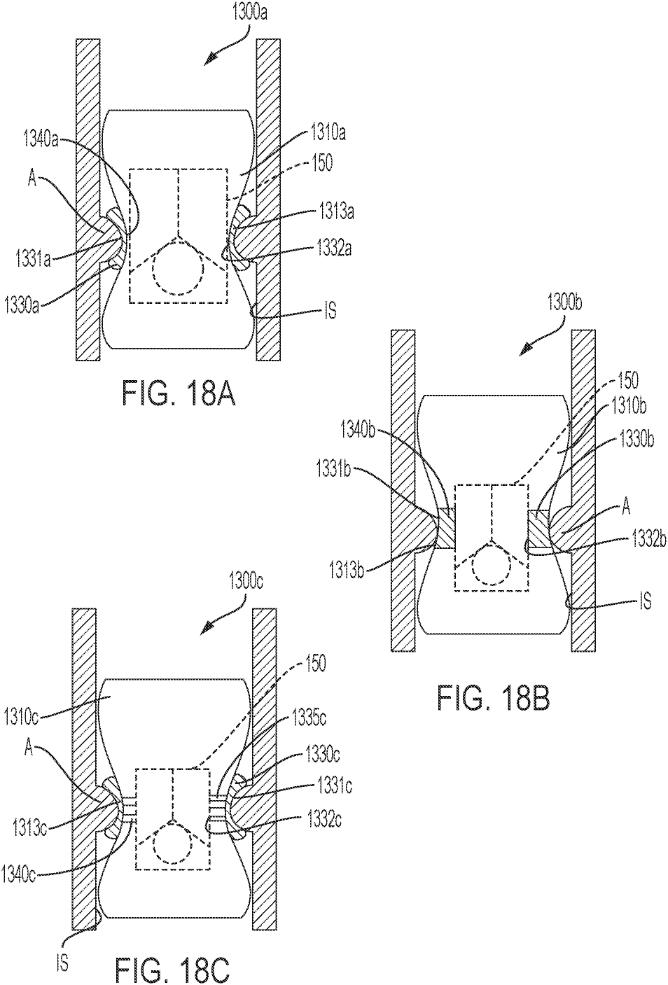

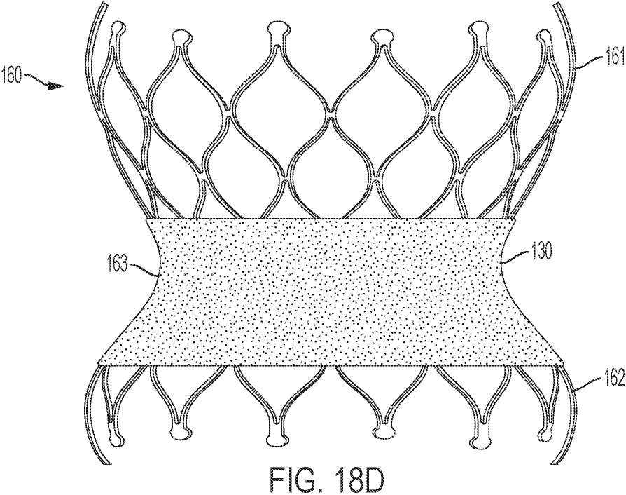

Inner and outer seal portions at a docking station waist portion may take a wide variety of forms. FIG. 18A schematically illustrates one exemplary embodiment of a docking station 1300a in which an impermeable sealing material 1330a is attached to an outer surface of the body 1310a at the waist portion 1313a and includes an outer seal portion 1331a that seals against a native annulus A of the internal surface IS, and an inner seal portion 1332a that seals against the installed valve 150 (e.g., through a latticed frame body at the valve seat 1340a).

FIG. 18B schematically illustrates another exemplary embodiment of a docking station 1300b in which an impermeable sealing material 1330b is attached to an inner surface of the body 1310b at the waist portion 1313b and includes an outer seal portion 1331b that seals against a native annulus A of the internal surface IS (e.g., through a latticed frame body), and an inner seal portion 1332b that seals against the installed valve 150 (e.g., at the valve seat 1340b, and optionally defining the valve seat). FIG. 18C schematically illustrates another exemplary embodiment of a docking station 1300c in which a first impermeable sealing material 1330c is attached to an outer surface of the body 1310c at the waist portion 1313c and includes an outer seal portion 1331c that seals against a native annulus A of the internal surface IS, and a second impermeable sealing material 1335c is attached to an inner surface of the body 1310c at the waist portion 1313c and includes an inner seal portion 1332c that seals against the installed valve 150 (e.g., at the valve seat 1340c, and optionally defining the valve seat).

Many annulus defining portions in a circulatory system, such as the tricuspid valve TV annulus, are not calcified and may not provide an optimal surface for sealing engagement with the docking station. According to some exemplary embodiments of the present disclosure, the sealing portion(s) of a docking station may include a valve sealing portion aligned with the valve (e.g., at the valve seat), and a tissue sealing portion aligned with either or both of the docking station end portions, spaced apart from the annulus of the internal surface IS, for example, for engagement with a more uniform, seal accommodating portion of the internal surface. The valve sealing portion and the tissue sealing portion may, but need not, be integral portions of a single sealing material.

FIG. 17B schematically illustrates an exemplary docking station 1200b including a sealing portion 1230b attached to the docking station body 1210b and extending from the waist portion 1213b of the body to the second (e.g., outflow) end portion 1212b, such that the sealing portion seals against the valve 150 at the waist portion 1213b (e.g., at the valve seat 1240b) and against the internal surface IS at the second end portion 1212b, proximal to the native annulus A. The sealing portion may additionally seal against the annulus A at the waist portion 1213b (e.g., using one of the sealing arrangements shown in FIGS. 18A - 18C and described above), for example, as a secondary seal location.

FIG. 17C schematically illustrates an exemplary docking station 1200c including a sealing portion 1230c attached to the docking station body 1210c and extending from the waist portion 1213c of the body to the first (e.g., inflow) end portion 1211c, such that the sealing portion seals against the valve 150 at the waist portion 1213c (e.g., at the valve seat 1240c) and against the internal surface IS at the first end portion 1211c distal to the native annulus A. The sealing portion may additionally seal against the annulus A at the waist portion 1213c (e.g., using one of the sealing arrangements shown in FIGS. 18A - 18C and described above), for example, as a secondary seal location.

FIG. 17D schematically illustrates an exemplary docking station 1200d including a sealing portion 1230d attached to the docking station body 1210d and extending from the first (e.g., inflow) end portion 1211d to the second (e.g., outflow) end portion 1212d, such that the sealing portion seals against the valve 150 at the waist portion 1213d (e.g., at the valve seat 1240d) and against the internal surface IS at the first and second end portions 1211d, 1212d distal and proximal to the native annulus A. The sealing portion may additionally seal against the annulus A at the waist portion 1213d (e.g., using one of the sealing arrangements shown in FIGS. 18A — 18C and described above), for example, as a secondary seal location.

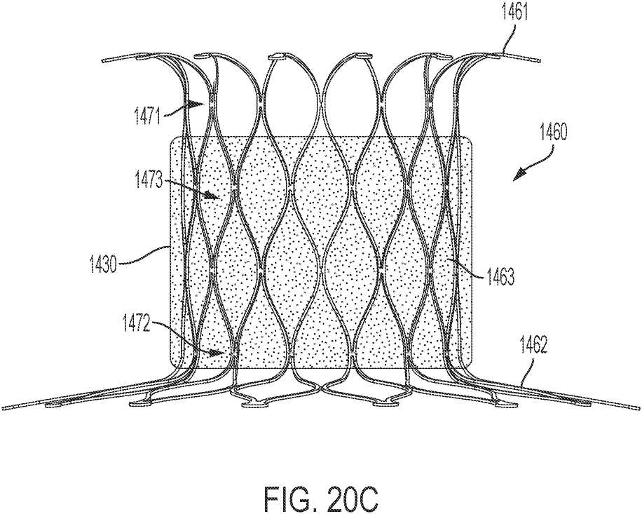

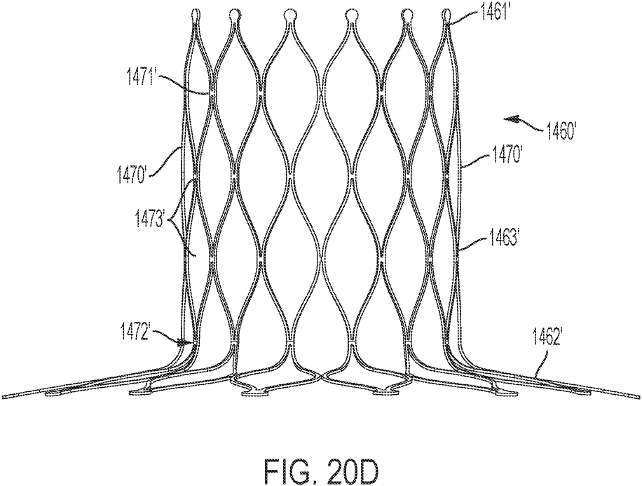

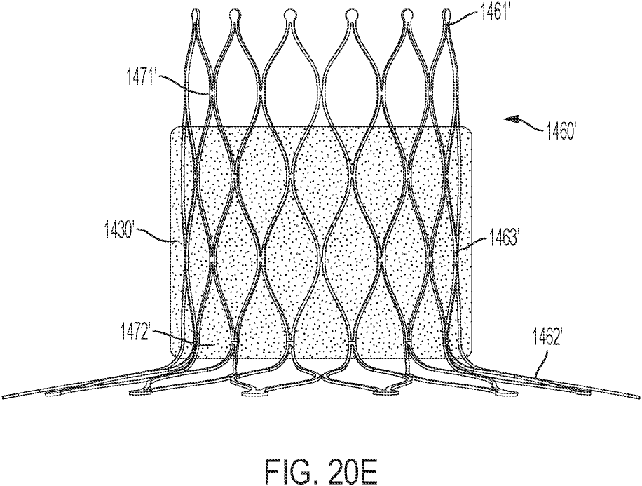

Outer seal portions, for example, at a docking station waist portion may take a wide variety of forms. As one example, a relatively thick strip or skirt of fabric material may be secured to an outer surface of the waist portion of the frame. This fabric material may be selected to be sufficiently impermeable to provide a seal between the frame and the native annulus at the deployment site, and may promote endothelialization over a period of time (e.g., up to about 30 days) from implantation. FIG. 18D illustrates one exemplary embodiment of an expandable frame 160 for a docking station, including a sealing skirt portion 130 secured to an outer surface of the waist portion 163 of the expandable frame 160. In an exemplary embodiment, the sealing skirt portion 130 includes a knitted polyethylene terephthalate (PET) with a thickness of at least about 0.25 mm, or about 0.4 mm, or 0.4 mm +/- 0.02 mm. At least in the short term, the knitted PET acts as a gasket to fill gaps between the frame and tissue, which creates a good seal and prevents leakage. In the long term (e.g., after up to about 30 days), the bulkier sealing material helps promote tissue ingrowth or endothelialization. While the expandable frame 160 of FIG. 18D is shown as being consistent with the expandable frame 160 of FIGS. 5A - 5D, the sealing outer skirt portion 130 may be provided with any of the expandable frames described herein, including, for example, the expandable frame 1460 of FIGS. 20A - 20B (as shown at 1430 in FIG. 20C), and the expandable frame 1460′ of FIG. 20D (as shown at 1430′ in FIG. 20E).

Where a docking station body includes an expandable lattice frame (e.g., any of the exemplary expandable frames described herein), the sealing portion (e.g., cloth/fabric) may be attached to selected ones of the strut-defining cells to provide a seal at one or more of the first end portion, the second end portion, and the waist portion of the frame. The sealing portion(s) may be formed from a variety of different suitable materials. As one example, an impermeable cloth or fabric sealing material may be utilized. The cloth may be selected to promote endothelialization, and may include, for example, one or more of high density polyethylene terephthalate (HDPET), expanded polytetrafluoroethylene (ePTFE), and electrospun polyurethane. In an exemplary arrangement, the cloth sealing material may be attached to the outer surface and/or the inner surface of the expandable frame using any of a variety of suitable attachment arrangements. For example, the cloth sealing material may be attached to the frame by sutures (e.g., Force Fiber® sutures by Teleflex Medical), adhesive (e.g., polyurethane), or other suitable arrangements. The cloth sealing material may be provided with a fiber orientation between about 30 degrees and about 60 degrees, for example, for ease of assembly. The sealing portion(s) may be formed by a single sealing material component (e.g., single sealing cloth) or by two or more sealing material components which may be secured in sealing engagement with each other (e.g., by sutures, stitches, adhesives, etc.). As one example, the sealing material may include a first sealing cloth ribbon attached to the inflow end portion and a second sealing cloth ribbon attached to the outflow end portion, with the two ribbons secured together (e.g., sewed together or bonded by adhesive) in sealing engagement at the waist portion of the frame.

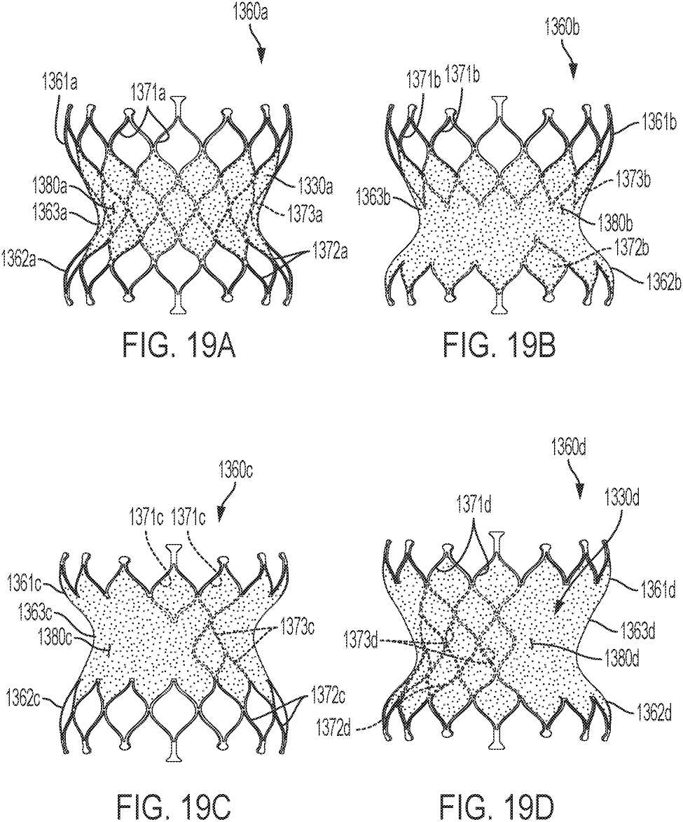

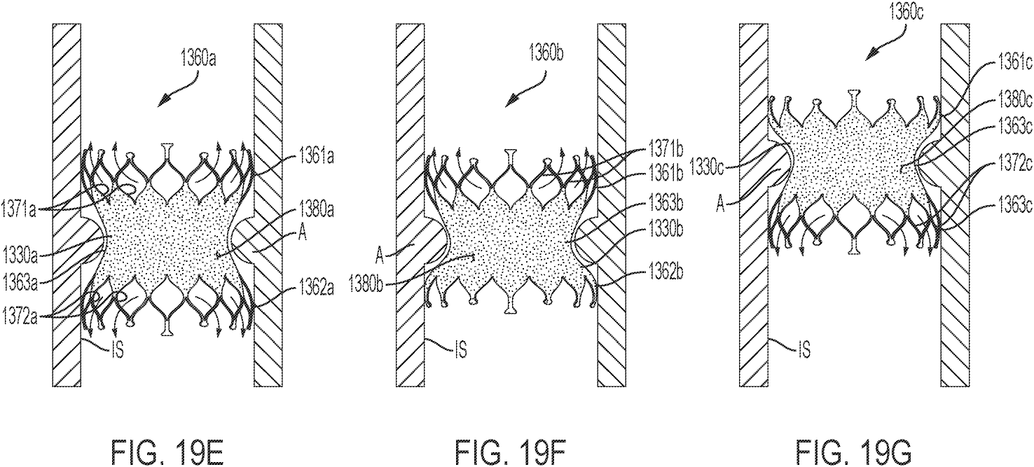

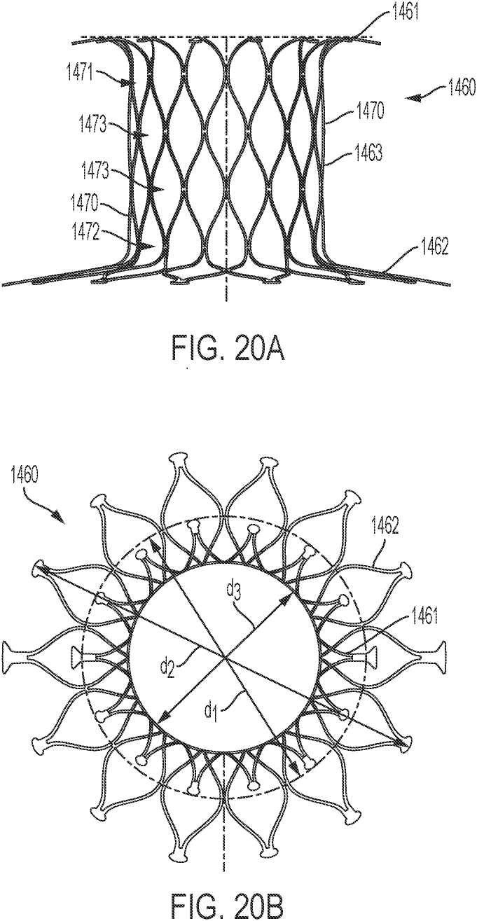

FIG. 19A illustrates an exemplary expandable frame 1360a including a sealing material (e.g., impermeable cloth) 1380a attached to the central cells 1373a to define a seal portion 1330a at the waist portion 1363a of the frame (similar to the embodiment of FIG. 17A), with the first and second end cells 1371a, 1372a uncovered to permit flow through a side portion of the first (e.g., inflow) and second (e.g., outflow) end portions 1361a, 1362a of the frame, as shown in FIG. 19E. As shown, the sealing material 1380a may be provided with a tooth-shaped pattern to fully cover the central cells 1373a. FIG. 19B illustrates an exemplary expandable frame 1360b including a sealing material (e.g., impermeable cloth) 1380b attached to the second end cells 1372b and the central cells 1373b to define a seal portion 1330b extending from the waist portion 1363b of the frame to the second end portion 1362b of the frame (similar to the embodiment of FIG. 17B), with the first end cells 1371b uncovered to permit flow through a side portion of the first (e.g., inflow) end portion 1361b of the frame, as shown in FIG. 19F. As shown, the sealing material 1380b may be provided with a tooth-shaped pattern to fully cover the second end cells 1372b and the central cells 1373b. FIG. 19C illustrates an exemplary expandable frame 1360c including a sealing material (e.g., impermeable cloth) 1380c attached to the first end cells 1371c and the central cells 1373c to define a seal portion 1330c extending from the waist portion 1363c of the frame to the first end portion 1361c of the frame (similar to the embodiment of FIG. 17C), with the second end cells 1372c uncovered to permit flow through the side of the second (e.g., outflow) end portion 1362c of the frame, as shown in FIG. 19G. As shown, the sealing material 1380c may be provided with a tooth-shaped pattern to fully cover the first end cells 1371c and the central cells 1373c. FIG. 19D illustrates an exemplary expandable frame 1360d including a sealing material (e.g., impermeable cloth) 1380d attached to the first end cells 1371d, the second end cells 1372d, and the central cells 1373d to define a seal portion 1330d extending from the first end portion 1361d of the frame across the waist portion 1363d to the second end portion 1362d of the frame (similar to the embodiment of FIG. 17D). As shown, the sealing material 1380d may be provided with a tooth-shaped pattern to fully cover the first end cells 1371d, the second end cells 1372d, and the central cells 1373d.

The sealing material 1380a-d of FIGS. 19A - 19D may include a variety of suitable materials. In an exemplary embodiment, a relatively thin (e.g., having a thickness of less than about 0.1 mm, or about 0.06 mm) knitted polyethylene terephthalate (PET) sealing fabric 1380a-d is secured to the frame. In some embodiments, a thinner sealing fabric (e.g., the sealing material 1380a-d of FIGS. 19A - 19D) is secured to an interior of the expandable frame, for example, to block the flow of blood through the frame lattice and to direct blood through the seated prosthetic valve, and a thicker sealing fabric (e.g., the sealing material 130 of FIG. 18D) is secured to the exterior of at least the waist portion of the expandable frame, as shown in FIG. 18D and described above, to provide a bulkier seal and/or tissue ingrowth in the annular space between the deployment site and the valve seat.

The valve seat can take a wide variety of different forms. In exemplary embodiments described herein, the valve seat is defined by the central cells of the expandable lattice frame at the narrowed waist portion of the frame. However, in other exemplary embodiments, the valve seat may be formed separately from the frame. The valve seat can take any form that provides a supporting surface for implanting or deploying a valve in the docking station after the docking station is implanted in the circulatory system. The valve is schematically illustrated herein to indicate that the valve can take a wide variety of different forms. For example, the valve may include a leaflet type THV, such as the Sapien 3 valve available from Edwards Lifesciences. In another exemplary embodiment, a THV may be integrally formed with the docking station thereby eliminating any seating engagement between the valve and the docking station frame. One or more features of other valves and valve arrangements may additionally or alternatively be used, including valves and valve arrangements described in the following references, the entire disclosures of each of which are incorporated herein by reference: U.S. Pat. No. 8,002,825, Published Patent Cooperation Treaty Application No. WO 2000/42950, U.S. Pat. No. 5,928,281, U.S. Pat. No. 6,558,418, U.S. Pat. No. 6,540,782, U.S. Pat. No. 3,365,728, U.S. Pat. No. 3,824,629, and U.S. Pat. No. 5,814,099.

Other features may additionally or alternatively be provided with the exemplary docking stations disclosed herein, in accordance with additional aspects of the present disclosure. For example, a docking station may be provided with one or more radiopaque markers, for example, for improved fluoroscopic visibility during the transcatheter procedures (e.g., implantation of the docking station and/or THV). In an exemplary embodiment, three or more radiopacque markers may be attached to a waist portion of a docking station. Many different attachment arrangements may be used. For example, the radiopaque markers may be sewn into pouches in the sealing material (e.g. cloth), for example, within one or more of the frame cells. As another example, the radiopaque markers may be press fit into the frame. Markers may include any suitable radiopaque material, including, for example, platinum-iridium, or a metal-infused polymer such as tantalum particle-infused polyurethane.

Still other expandable frame arrangements may be used for deployment at locations in the circulatory system including a non-calcified annulus or other surface, variations in internal surface contours, or other such characteristics, such as, for example, the tricuspid valve TV region between the right atrium RA and the right ventricle RV. FIGS. 20A and 20B illustrate an exemplary expandable frame 1460 including a distal or first end flange portion 1461 extending primarily radially outward to a first major lateral dimension (e.g., diameter) d₁ in the unconstrained condition, a proximal or second end flange portion 1462 extending primarily radially outward to a second major lateral dimension (e.g., diameter) d₂ in the unconstrained condition, and a narrower, substantially axial (e.g., cylindrical) waist portion 1463 having a third major lateral dimension (e.g., diameter) d₃. The flange portions 1461, 1462 may extend primarily radially outward or substantially perpendicularly (e.g., about 70° to about 110°) with respect to a central axis of the frame when in the unconstrained condition. In the illustrated example, the first end flange portion 1461 extends at a slightly acute angle with respect to the axially extending waist portion 1463, and the second end flange portion 1462 extends at a slightly obtuse angle with respect to the axial portion 1463. The primarily radial flange portions may be disposed at other angles, including, for example, about 60° to about 120° with respect to the frame central axis.

As shown, the frame 1460 may include a plurality of struts 1470 forming one or more rows of first end cells 1471, second end cells 1472, and central cells 1473. As shown, the first and second end cells 1471, 1472 may extend across the bent portions between the first and second end flange portions 1461, 1462 and the cylindrical waist portion 1463.

In the crimped or compressed condition (e.g., when stored in a catheter), the first end flange portion 1461 may extend axially in the distal direction, substantially collinear to the waist portion 1463, and the second end flange portion 1462 may extend axially in the proximal direction, substantially collinear to the waist portion 1463. When deployed at an interior surface of the circulatory system, the first end flange portion 1461 may bend radially outward to engage an internal surface distal to a native annulus (e.g., the tricuspid valve annulus), and the second end flange portion 1462 may bend radially outward to engage an internal surface proximal to the native annulus. In the deployed condition, engagement of the first and second flange portions 1461, 1462 with the internal surface may constrain either or both of the first and second flange portions from fully bending to the unconstrained condition. This flexed condition of the deployed frame flange portions 1461, 1462 may provide desired retaining forces of the frame 1460 against the internal surface, while maintaining a radial gap between the waist portion 1463 and the native annulus.

While the diameters d₁, d₂ of the first and second end flange portions 1461, 1462 may be substantially equal in size, in other embodiments, one of the first and second end portions may have an outer radial portion that is larger than the outer radial portion of the other end portion. In the exemplary expandable frame 1460 of FIGS. 20A - 20B, the second end (e.g., outflow) flange portion 1462 has an outer diameter d₂ larger than (e.g., up to about 50% larger than) an outer diameter d₁ of the first end (inflow) flange portion 1461, for example to anchor the docking station primarily or entirely to the right ventricle when the docking station is installed at the tricuspid valve annulus. The primarily radial or substantially perpendicular second end flange portion 1462 allows the apices of the flange portion to engage the right ventricle wall without engaging (and potentially damaging) the chordae tendineae within the right ventricle. In other applications, an expandable frame may have a first end flange portion larger than the second flange end portion, or the first and second end flange portion sizes may differ to varying degrees.

In another embodiment, an expandable frame may be provided with a primarily radially extending flange on only one end of the frame, with the substantially axially extending waist portion extending primarily or entirely axially to the other end of the frame. FIG. 20D illustrates an exemplary expandable frame 1460′ including a second end flange portion 1462′ extending primarily radially outward in the unconstrained condition, and a proximal end portion 1461′ extending primarily or entirely axially from a substantially axially extending waist portion 1463′ of the frame 1460′. The flange portion 1462′ may extend primarily radially outward or substantially perpendicularly (e.g., about 70° to about 110°) with respect to a central axis of the frame when in the unconstrained condition. In the illustrated example, the flange portion 1462′ extends at a slightly obtuse angle with respect to the waist portion 1463′. The primarily radial flange portion may be disposed at other angles, including, for example, about 60° to about 120° with respect to the frame central axis.

As shown, the frame 1460′ may include a plurality of struts 1470′ forming one or more rows of first end cells 1471′, second end cells 1472′, and central cells 1473′. As shown, the second end cells 1472′ may extend across the bent portions between the flange portion 1462′ and the waist portion 1463′.

In the crimped or compressed condition (e.g., when stored in a catheter), the second end flange portion 1462′ may extend axially in the proximal direction, substantially collinear to the waist portion 1463′. When deployed at an interior surface of the circulatory system, the second end flange portion 1462′ may bend radially outward to engage an internal surface proximal to the native annulus. In the deployed condition, engagement of the flange portion 1462′ with the internal surface may constrain either or both of the first and second flange portions from fully bending to the unconstrained condition. This flexed condition of the deployed frame flange portion 1462′ may provide desired retaining forces of the frame 1460′ against the internal surface, while maintaining a radial gap between the waist portion 1463′ and the native annulus.

The primarily radial or substantially perpendicular second end flange portion 1462′ allows the apices of the flange portion to engage the right ventricle wall without engaging (and potentially damaging) the chordae tendineae within the right ventricle. In other applications, an expandable frame may have only a first end flange portion, with no second flange end portion.

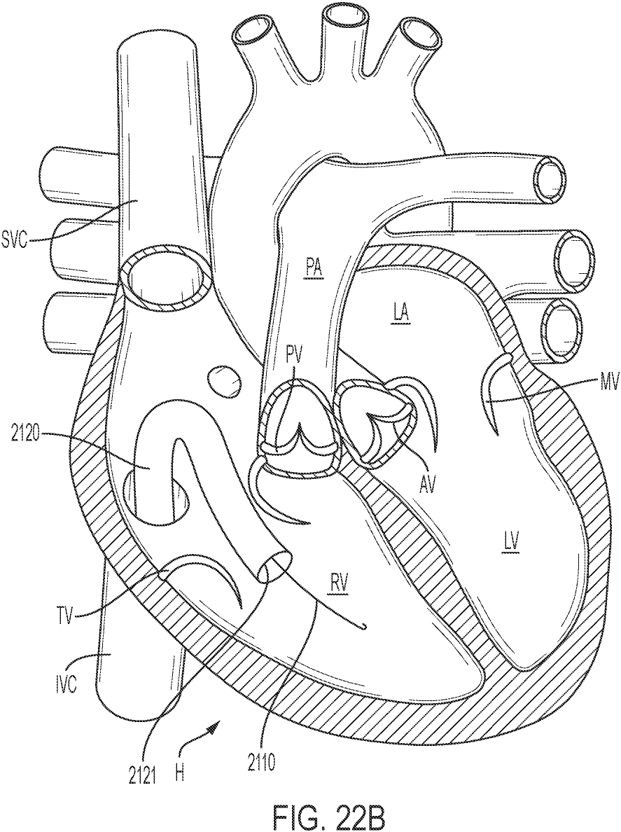

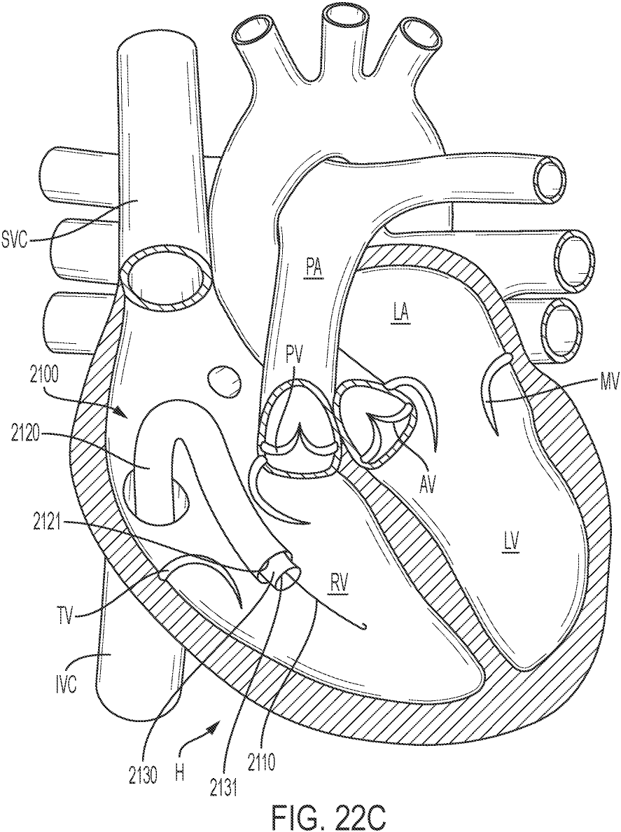

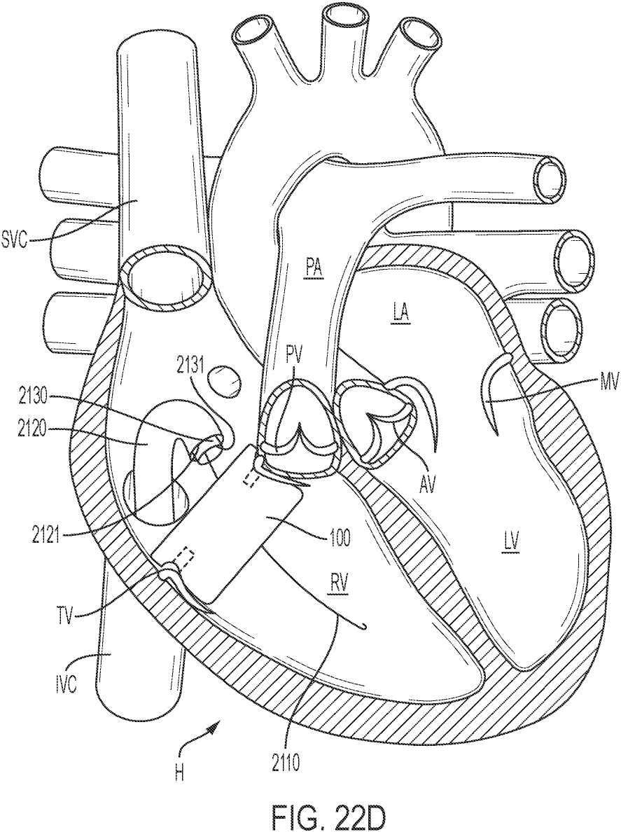

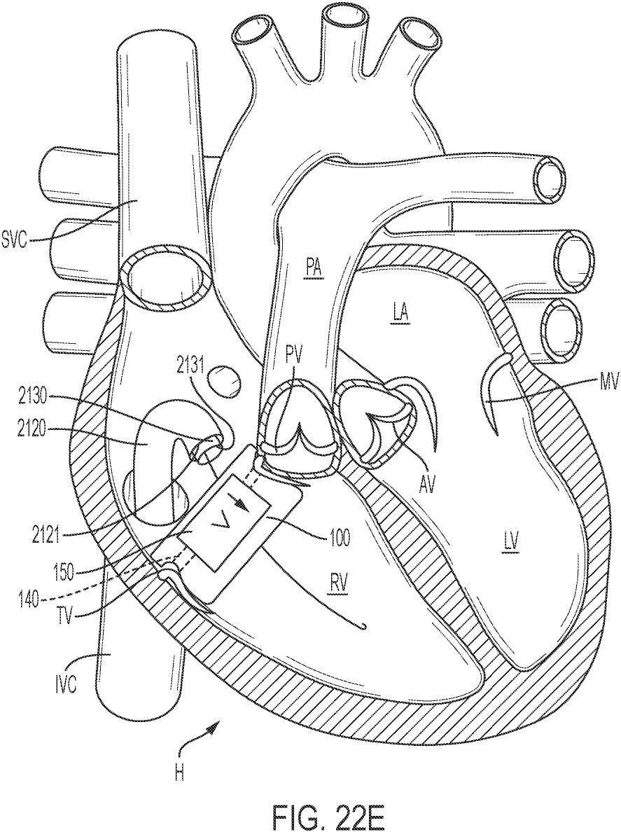

Methods of treating a patient (e.g., methods of treating heart valve dysfunction, regurgitation, etc.) may include a variety of steps, including steps associated with introducing and deploying a docking station and transcatheter heart valve THV in a desired location/treatment area and introducing and deploying a valve in the docking station. The docking station and prosthetic valve can be positioned and deployed in a wide variety of different ways. For example, FIGS. 21A - 21G illustrate a docking station 100 (e.g., any of the exemplary docking stations described herein) and prosthetic tricuspid valve 150 being sequentially deployed by a catheter system 2000 from the superior vena cava SVC. In the illustrated method, a guide wire 2010 is inserted through the superior vena cava SVC, right atrium RA and tricuspid valve TV, and into the right ventricle RV (FIG. 21A). An outer catheter 2020 is then guided, using the guide wire 2010, through the superior vena cava SVC, right atrium RA and tricuspid valve TV, and into the right ventricle RV (FIG. 21B). In other exemplary methods, the catheter may be guided into the right ventricle RV without the use of a guide wire. A first, docking station deploying inner catheter 2030 is then guided within the outer catheter 2020 to extend an open end 2031 of the first inner catheter to (or beyond) the open end 2021 of the outer catheter (FIG. 21C). The outer and first inner catheters 2020, 2030 are then adjusted to align the open end 2031 of the first inner catheter 2030 with the intended deployment site for the docking station 100, and the compressed docking station 100 is guided through and out of the first inner catheter 2030, with the docking station expanding (e.g., self-expanding or manually expanded, such as with a balloon) into retaining and sealing engagement with the deployment site (FIG. 21D). The first inner catheter 2030 is then withdrawn from the outer catheter 2020 (FIG. 21E), and a second, valve deploying inner catheter 2040 is guided within the outer catheter 2020 to extend an open end 2041 of the second inner catheter to (or beyond) the open end 2021 of the outer catheter 2020 (FIG. 21F). The outer and second inner catheters 2020, 2040 are then adjusted to align the open end 2041 of the second inner catheter with the intended deployment site for the valve 150, and the compressed valve 150 is guided through and out of the second inner catheter 2040, with the valve expanding (e.g., self-expanding or manually expanded, such as with a balloon) into retaining and sealing engagement with the valve seat 140 (FIG. 21G). The second inner catheter 2040, outer catheter 2020, and guide wire 2010 may then be withdrawn through the superior vena cava SVC. In other exemplary methods, the first inner catheter 2030 may be used to install both the docking station 100 and the valve 150, without the use of a separate second inner catheter, similar to the method described below and shown in FIGS. 22A - 22E.

As another example, FIGS. 22A - 22E illustrate a docking station 100 (e.g., any of the exemplary docking stations described herein) and prosthetic tricuspid valve 150 being sequentially deployed by a catheter system 2100 from the inferior vena cava IVC. In the illustrated method, a guide wire 2110 is inserted through the inferior vena cava IVC, right atrium RA and tricuspid valve TV, and into the right ventricle RV (FIG. 22A). An outer catheter 2120 is then guided, using the guide wire 2110, through the inferior vena cava IVC, right atrium RA and tricuspid valve TV, and into the right ventricle RV (FIG. 22B). In other exemplary methods, the catheter may be guided into the right ventricle RV without the use of a guide wire. An inner catheter 2130 is then guided within the outer catheter 2120 to extend an open end 2131 of the first inner catheter to (or beyond) the open end 2121 of the outer catheter (FIG. 22C). The outer and inner catheters 2120, 2130 are then adjusted to align the open end 2131 of the first inner catheter 2130 with the intended deployment site for the docking station, and the compressed docking station 100 is guided through and out of the first inner catheter 2130, with the docking station expanding (e.g., self-expanding or manually expanded) into retaining and sealing engagement with the deployment site (FIG. 22D). The outer and inner catheters 2120, 2130 are then adjusted to align the open end 2131 of the inner catheter with the intended deployment site for the valve, and the compressed valve 150 is guided through and out of the inner catheter 2130, with the valve expanding (e.g., self-expanding or manually expanded) into retaining and sealing engagement with the valve seat 140 (FIG. 22E). The inner catheter 2130, outer catheter 2120, and guide wire 2110 may then be withdrawn through the inferior vena cava IVC. In other exemplary methods, a second inner catheter may be used to install the valve 150, similar to the method described above and shown in FIGS. 21A - 21G.

### Additional Examples

Example 1. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion;
wherein the expandable frame includes a plurality of struts extending between first apices at the first end portion to second apices at the second end portion, wherein at least one of the first apices and the second apices are contoured radially inward.

Example 2. The expandable frame of Example 1, wherein the second major lateral dimension is greater than the first major lateral dimension.

Example 3. The expandable frame of Example 1, wherein the first major lateral dimension is greater than the second major lateral dimension.

Example 4. The expandable frame of Example 1, wherein the first major lateral dimension is substantially equal to the first major lateral dimension.

Example 5. The expandable frame of any of Examples 1-4, wherein a first axial length from an axial midpoint of the waist portion to the first apices is greater than a second axial length from the axial midpoint of the waist portion to the second apices.

Example 6. The expandable frame of any of Examples 1-4, wherein a first axial length from an axial midpoint of the waist portion to the first apices is substantially equal to a second axial length from the axial midpoint of the waist portion to the second apices.

Example 7. The expandable frame of any of Examples 1-6, wherein the first outer radial portion has a cross-sectional shape substantially the same as a cross-sectional shape of the second outer radial portion.

Example 8. The expandable frame of any of Examples 1-6, wherein the first outer radial portion has a cross-sectional shape different than a cross-sectional shape of the second outer radial portion.

Example 9. The expandable frame of any of Examples 1-8, wherein the first outer radial portion has a cross-sectional shape substantially the same as a cross-sectional shape of the inner radial portion.

Example 10. The expandable frame of any of Examples 1-8, wherein the first outer radial portion has a cross-sectional shape different than a cross-sectional shape of the inner radial portion.

Example 11. The expandable frame of any of Examples 1-10, wherein the first outer radial portion has a cross-sectional shape that is one of: circular, elliptical, D-shaped, and rounded D-shaped.

Example 12. The expandable frame of any of Examples 1-10, wherein the second outer radial portion has a cross-sectional shape that is one of: circular, elliptical, D-shaped, and rounded D-shaped.

Example 13. The expandable frame of any of Examples 1-10, wherein the inner radial portion has a cross-sectional shape that is one of: circular, elliptical, D-shaped, and rounded D-shaped.

Example 14. The expandable frame of any of Examples 1-13, wherein an axial midpoint of the waist portion is concave.

Example 15. The expandable frame of any of Examples 1-13, wherein an axial midpoint of the waist portion has a substantially straight axially extending profile.

Example 16. The expandable frame of any of Examples 1-15, wherein the first end portion of the frame comprises at least one row of first end cells defined by the plurality of struts, the second end portion of the frame comprises at least one row of second end cells defined by the plurality of struts, and the waist portion of the frame comprises at least one row of central cells defined by the plurality of struts.

Example 17. The expandable frame of any of Examples 1-16, wherein the plurality of struts include first end strut portions defining the first end portion of the frame, second end strut portions defining the second end portion of the frame, and central strut portions defining the waist portion of the frame.

Example 18. The expandable frame of Example 17, wherein the central strut portions have a cross-sectional area greater than a cross-sectional area of the first and second end strut portions.

Example 19. The expandable frame of any of Examples 1-18, wherein the other of the first apices and the second apices are contoured radially inward.

Example 20. The expandable frame of any of Examples 1-18, wherein the other of the first apices and the second apices are contoured radially outward.

Example 21. The expandable frame of any of Examples 1-20, wherein the expandable frame is sized to be implanted at a tricuspid valve of a human heart, with the first end portion retained in a right atrium, the second end portion retained in a right ventricle, and the waist portion aligned with the tricuspid valve.

Example 22. The expandable frame of any of Examples 1-21, wherein the first major lateral dimension is approximately 50 mm.

Example 23. The expandable frame of any of Examples 1-22, wherein the third major lateral dimension is approximately 27 mm.

Example 24. The expandable frame of any of Examples 1-23, further comprising at least one radiopaque marker attached to the frame.

Example 25. The expandable frame of Example 23, wherein the at least one radiopaque marker is attached to the waist portion of the frame.

Example 26. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion;
wherein the expandable frame includes a plurality of struts extending between first apices at the first end portion to second apices at the second end portion, wherein the plurality of struts include first end strut portions defining the first end portion of the frame, second end strut portions defining the second end portion of the frame, and central strut portions defining the waist portion of the frame;
wherein the central strut portions have a cross-sectional area greater than a cross-sectional area of the first and second end strut portions.

Example 27. An expandable frame for a docking station configured to retain and position a transcatheter heart valve in a circulatory system, the expandable frame comprising:
an enlarged first end portion having an elliptical first outer radial portion with a first major lateral dimension, an enlarged second end portion having an elliptical second outer radial portion with a second major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion.

Example 28. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion;
wherein a first axial length from an axial midpoint of the waist portion to an edge of the first end portion is greater than a second axial length from the axial midpoint of the waist portion to an edge of the second end portion.

Example 29. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension greater than the first major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion.

Example 30. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions, wherein the first outer radial portion has a cross-sectional shape different than a cross-sectional shape of at least one of the second outer radial portion and the inner radial portion;
a retaining portion at least partially defined by at least one of the first and second end portions; and
a valve seat at least partially defined by the waist portion.

Example 31. The expandable frame of any of Examples 27-30, wherein the expandable frame includes a plurality of struts extending between first apices at the first end portion to second apices at the second end portion, wherein the plurality of struts include first end strut portions defining the first end portion of the frame, second end strut portions defining the second end portion of the frame, and central strut portions defining the waist portion of the frame.

Example 32. The expandable frame of any of Examples 26-31, wherein the first outer radial portion has a cross-sectional shape that is one of: circular, elliptical, D-shaped, and rounded D-shaped.

Example 33. The expandable frame of any of Examples 26-32, wherein the second outer radial portion has a cross-sectional shape that is one of: circular, elliptical, D-shaped, and rounded D-shaped.

Example 34. The expandable frame of any of Examples 26-33, wherein the inner radial portion has a cross-sectional shape that is one of: circular, elliptical, D-shaped, and rounded D-shaped.

Example 35. The expandable frame of any of Examples 26-34, wherein an axial midpoint of the waist portion is concave.

Example 36. The expandable frame of any of Examples 26-34, wherein an axial midpoint of the waist portion has a substantially straight axially extending profile.

Example 37. The expandable frame of any of Examples 26-36, wherein the expandable frame is sized to be implanted at a tricuspid valve of a human heart, with the first end portion retained in a right atrium, the second end portion retained in a right ventricle, and the waist portion aligned with the tricuspid valve.

Example 38. The expandable frame of any of Examples 26-37, wherein the first major lateral dimension is approximately 50 mm.

Example 39. The expandable frame of any of Examples 26-38, wherein the third major lateral dimension is approximately 27 mm.

Example 40. The expandable frame of any of Examples 26-39, further comprising at least one radiopaque marker attached to the frame.

Example 41. The expandable frame of Example 40, wherein the at least one radiopaque marker is attached to the waist portion of the frame.

Example 42. A docking station configured to retain and position a transcatheter heart valve in a circulatory system, the docking station comprising
the expandable frame of any of Examples 1-41; and
a sealing portion including a sealing material at least partially disposed on the waist portion, the sealing portion providing a seal between the expandable frame and a deployment site of a circulatory system when the docking station is implanted at the deployment site.

Example 43. The docking station of Example 42, wherein the sealing material is at least partially disposed on the first end portion of the frame.

Example 44. The docking station of any of Examples 42 and 43, wherein the sealing material is at least partially disposed on the second end portion of the frame.

Example 45. The docking station of any of Examples 42-44, wherein the sealing material is secured to an external surface of the frame.

Example 46. The docking station of any of Examples 42-45, wherein the sealing material is secured to an internal surface of the frame.

Example 47. The docking station of any of Examples 42-46, wherein the sealing material comprises at least one of: an impermeable cloth, a foam, and a tissue.

Example 48. The docking station of any of Examples 42-47, wherein the sealing material comprises first and second sealing material components.

Example 49. The docking station of Example 48, wherein the first and second sealing material components are secured together at the waist portion of the frame.

Example 50. The docking station of any of Examples 42-49, wherein the sealing material comprises an outer fabric material secured to an outer surface of the expandable frame.

Example 51. The docking station of Example 50, wherein the outer fabric material comprises a knitted PET material.

Example 52. The docking station of any of Examples 50 and 51, wherein the outer fabric material has a thickness of at least about 0.25 mm.

Example 53. The docking station of any of Examples 42-52, wherein the sealing material comprises an inner fabric material secured to an inner surface of the expandable frame.

Example 54. The docking station of Example 53, wherein the inner fabric material comprises a woven PET material.

Example 55. The docking station of any of Examples 53 and 54, wherein the inner fabric material has a thickness of less than about 0.1 mm.

Example 56. The docking station of any of Examples 42-55, wherein the first end portion of the frame comprises at least one row of first end cells defined by the plurality of struts, the second end portion of the frame comprises at least one row of second end cells defined by the plurality of struts, and the waist portion of the frame comprises at least one row of central cells defined by the plurality of struts.

Example 57. The docking station of Example 56, wherein at least one of the first end cells is uncovered to permit flow through a side portion of the first end portion.

Example 58. The docking station of any of Examples 56 and 57, wherein at least one of the second end cells is uncovered to permit flow through a side portion of the second end portion.

Example 59. An expandable frame for a docking station configured to retain and position a transcatheter heart valve in a circulatory system, the expandable frame comprising:
a first end flange portion extending radially outward to a first outer radial portion with a first major lateral dimension, an enlarged second end portion extending radially outward to a second outer radial portion with a second major lateral dimension, and a narrowed axially extending central waist portion having a third major lateral dimension smaller than the first and second major lateral dimensions, with the first and second end flange portions extending substantially perpendicularly to a central axis of the frame when the frame is in an unconstrained condition;
a retaining portion at least partially defined by at least one of the first and second end flange portions; and
a valve seat at least partially defined by the waist portion.

Example 60. An expandable frame for a docking station configured to retain and position a transcatheter heart valve in a circulatory system, the expandable frame comprising:
a first end flange portion extending radially outward to a first outer radial portion with a first major lateral dimension, a narrowed substantially axially extending central waist portion having a second major lateral dimension smaller than the first major lateral dimensions, and a second end portion extending substantially axially from the narrowed substantially axially extending central waist portion, with the first end flange portion extending substantially perpendicularly to a central axis of the frame when the frame is in an unconstrained condition;
a retaining portion at least partially defined by the first end flange portion; and
a valve seat at least partially defined by the waist portion.

Example 61. A docking station configured to retain and position a transcatheter heart valve in a circulatory system, the docking station comprising:
the expandable frame of any of Examples 59 and 60; and
a sealing portion including a sealing material at least partially disposed on the waist portion, the sealing portion providing a seal between the expandable frame and a deployment site of a circulatory system when the docking station is implanted at the deployment site.

Example 62. The docking station of Example 61, wherein the sealing material comprises an outer fabric material secured to an outer surface of the expandable frame.

Example 63. The docking station of Example 62, wherein the outer fabric material comprises a knitted PET material.

Example 64. The docking station of any of Examples 62 and 63, wherein the outer fabric material has a thickness of at least about 0.25 mm.

Example 65. The docking station of any of Examples 61-64, wherein the sealing material comprises an inner fabric material secured to an inner surface of the expandable frame.

Example 66. The docking station of Example 65, wherein the inner fabric material comprises a woven PET material.

Example 67. The docking station of any of Examples 65 and 66, wherein the inner fabric material has a thickness of less than about 0.1 mm.

Example 68. A method of deploying a docking station to a tricuspid valve of a human heart, the method comprising:
guiding an outer catheter through a right atrium and tricuspid valve, and into a right ventricle;
guiding an inner catheter within the outer catheter to extend an open end of the inner catheter to or beyond an open end of the outer catheter;
adjusting the outer and inner catheters to align the open end of the inner catheter with an intended deployment site for a docking station; and
guiding a compressed docking station through and out of the inner catheter, with the docking station expanding into retaining and sealing engagement with the deployment site.

Example 69. The method of Example 67, wherein the docking station comprises the docking station of any of Examples 42-58 and 61-67.

Any one or more of the exemplary docking stations and expandable frame arrangements described herein may be used in the above described methods. One or more features of other docking stations and expandable frame arrangements may additionally or alternatively be used, including docking stations and/or expandable frames described in the following references, the entire disclosures of each of which are incorporated herein by reference: U.S. Pat. Application Publication No. 2019/0000615, and U.S. Pat. No. 10,363,130.

The foregoing primarily describes embodiments of docking stations that are self-expanding. But the docking stations shown and described herein can be modified for delivery of balloon-expandable and/or mechanically-expandable docking devices, within the scope of the present disclosure. That is to say, delivering balloon-expandable and/or mechanically-expandable docking stations to an implantation location can be performed percutaneously.

In view of the many possible embodiments to which the principles of the disclosed invention may be applied, it should be recognized that the illustrated embodiments are only preferred examples of the invention and should not be taken as limiting the scope of the invention. All combinations or subcombinations of features of the foregoing exemplary embodiments are contemplated by this application. The scope of the invention is defined by the following claims. We therefore claim as our invention all that comes within the scope and spirit of these claims.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions--such as alternative materials, structures, configurations, methods, circuits, devices and components, alternatives as to form, fit and function, and so on--may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to include both the specified value values within 5% of the specified value, and values within 10% of the specified value, unless expressly stated otherwise. Further, it is to be understood that the drawings accompanying the present disclosure may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

1. An expandable frame for a docking station configured to retain and position a transcatheter heart valve in a circulatory system, the expandable frame comprising:
an enlarged first end portion having a first outer radial portion with a first major lateral dimension, an enlarged second end portion having a second outer radial portion with a second major lateral dimension, and a narrowed central waist portion having an inner radial portion with a third major lateral dimension smaller than the first and second major lateral dimensions;
a retaining portion at least partially defined by at least one of the first and second end portions; and
a valve seat at least partially defined by the waist portion;
wherein the expandable frame includes a plurality of struts extending between first apices at the first end portion to second apices at the second end portion, wherein at least one of the first apices and the second apices are contoured radially inward.

2. The expandable frame of claim 1, wherein the second major lateral dimension is greater than the first major lateral dimension.

3. The expandable frame of claim 1, wherein the first major lateral dimension is greater than the second major lateral dimension.

4. The expandable frame of claim 1, wherein the first major lateral dimension is substantially equal to the first major lateral dimension.

5. The expandable frame of claim 1, wherein a first axial length from an axial midpoint of the waist portion to the first apices is greater than a second axial length from the axial midpoint of the waist portion to the second apices.

6. The expandable frame of claim 1, wherein a first axial length from an axial midpoint of the waist portion to the first apices is substantially equal to a second axial length from the axial midpoint of the waist portion to the second apices.

7. The expandable frame of claim 1, wherein the first outer radial portion has a cross-sectional shape substantially the same as a cross-sectional shape of the second outer radial portion.

8. The expandable frame of claim 1, wherein the first outer radial portion has a cross-sectional shape different than a cross-sectional shape of the second outer radial portion.

9. The expandable frame of claim 1, wherein the first outer radial portion has a cross-sectional shape substantially the same as a cross-sectional shape of the inner radial portion.

10. The expandable frame of claim 1, wherein the first outer radial portion has a cross-sectional shape different than a cross-sectional shape of the inner radial portion.

11. The expandable frame of claim 1, wherein the first outer radial portion has a cross-sectional shape that is one of: circular, elliptical, D-shaped, and rounded D-shaped.

12. The expandable frame of claim 1, wherein the second outer radial portion has a cross-sectional shape that is one of: circular, elliptical, D-shaped, and rounded D-shaped.

13. The expandable frame of claim 1, wherein the inner radial portion has a cross-sectional shape that is one of: circular, elliptical, D-shaped, and rounded D-shaped.

14. The expandable frame of claim 1, wherein an axial midpoint of the waist portion is concave.

15. The expandable frame of claim 1, wherein an axial midpoint of the waist portion has a substantially straight axially extending profile.

16. The expandable frame of claim 1, wherein the first end portion of the frame comprises at least one row of first end cells defined by the plurality of struts, the second end portion of the frame comprises at least one row of second end cells defined by the plurality of struts, and the waist portion of the frame comprises at least one row of central cells defined by the plurality of struts.

17. The expandable frame of claim 1, wherein the plurality of struts include first end strut portions defining the first end portion of the frame, second end strut portions defining the second end portion of the frame, and central strut portions defining the waist portion of the frame.

18. The expandable frame of claim 17, wherein the central strut portions have a cross-sectional area greater than a cross-sectional area of the first and second end strut portions.

19. The expandable frame of claim 1, wherein the other of the first apices and the second apices are contoured radially inward.

20. The expandable frame of claim 1, wherein the other of the first apices and the second apices are contoured radially outward.

21. The expandable frame of claim 1, wherein the expandable frame is sized to be implanted at a tricuspid valve of a human heart, with the first end portion retained in a right atrium, the second end portion retained in a right ventricle, and the waist portion aligned with the tricuspid valve.

22. The expandable frame of claim 1, wherein the first major lateral dimension is approximately 50 mm.

23. The expandable frame of claim 1, wherein the third major lateral dimension is approximately 27 mm.

24. The expandable frame of claim 1, further comprising at least one radiopaque marker attached to the frame.

25. The expandable frame of claim 23, wherein the at least one radiopaque marker is attached to the waist portion of the frame.
